(19) United States
(12) Patent Application Publication
(10) Pub. No.: US 2009/0171241 A1
(43) Pub. Date: Jul. 2, 2009

(54) SYSTEM AND METHOD FOR URINARY TRACT CELL COLLECTION, DIAGNOSIS, AND CHEMOTHERAPY

(76) Inventors: Maurice M. Garcia, San Francisco, CA (US); Peter R. Carroll, San Francisco, CA (US)

Correspondence Address: JOHN P. O'BANION;O'BANION & RITCHEY LLP, 400 CAPITOL MALL SUITE 1550, SACRAMENTO, CA, 95814, US

(21) Appl. No.: 12/203,864

(22) Filed: Sep. 3, 2008

Related U.S. Application Data
(63) Continuation of application No. PCT/US2007/063209, filed on Mar. 2, 2007.
(60) Provisional application No. 60/778,750, filed on Mar. 3, 2006.

(51) Int. Cl. A61B 10/02

(52) U.S. Cl. 600/563

(57) ABSTRACT

A system and method delivers and collects a wash medium into a lumen of the urinary tract system. Isolated saline delivery washes cells from a urethra that are collected for cancer diagnosis. BCG is delivered for local chemotherapy. The isolated delivery and collection is from a region bound by a distal occlusion and proximal occlusion. Inflatable balloons provide the occlusion and or a penis clamp may be used. The two balloons may telescope relative to each other. A balloon inflation lumen may collapse a valve membrane to occlude distal flow of infused fluid to only proximal of the balloon.

### CROSS-REFERENCE TO RELATED APPLICATIONS

This application claims priority from, and is a 35 U.S.C. § 111(a) continuation of, co-pending PCT international application serial number PCT/US2007/063209, filed on Mar. 2, 2007, incorporated herein by reference in its entirety, which claims priority from U.S. Provisional Patent Application Ser. No. 60/778,750, filed on Mar. 3, 2006, incorporated herein by reference in its entirety, and also claims priority from U.S.

Provisional Patent Application Ser. No. 60/802,316, filed on May 19, 2006, incorporated herein by reference in its entirety.

### STATEMENT REGARDING FEDERALLY SPONSORED RESEARCH OR DEVELOPMENT

Not Applicable

### BACKGROUND OF THE INVENTION

1. Field of the Invention

This invention relates to the field of medical devices. More specifically, it relates to a system, apparatus, and method for diagnosing and treating cancer in a patient, and still more particularly as it relates to the patient's urethra.

2. Description of Related Art

Cancer as it relates to the urinary tract is a significant and potentially deadly medical condition. The ability to effectively diagnose this condition, especially in early stages, and to treat it, remains significant unmet needs in clinical medicine and patient care. Moreover, available treatments often carry the risk of recurrence, which also need early detection for most favorable re-treatment results.

In one particular regard, patients who have had bladder cancer, and have had their diseased bladder replaced with a “neobladder” (new bladder) made from their own intestine, are still at risk for recurrence of “bladder cancer” disease, even if they already had their bladder removed. The entire urinary system—from the inside lining of the kidneys, where urine is made, through the ureters, which connect the kidney to the bladder, through the urethra, which connects the bladder to the outside world—is all coated with the same tissue: urothelium. The term “urethral recurrence” thus generally indicates the recurrence of bladder cancer in the urethra.

The bladder is generally considered the most common area of the urinary system where people will have cancer of the urothelium, i.e., “bladder cancer,” occur. However, as noted above, bladder cancer can occur anywhere along the urinary tract. Therefore, when a patient has bladder cancer, and ultimately has their diseased bladder removed (even when only the bladder contained cancer), they are still at risk for developing “bladder cancer” elsewhere. This includes the kidneys and ureter (located up-stream from the bladder), and the urethra (located down-stream from the bladder). In fact, because these patients have already had bladder cancer, they are at much higher risk than the average person for having the cancer “develop” elsewhere (e.g., in the kidneys, ureter, and urethra). So, these patients require careful “monitoring” for the remainder of their lives.

Conventional monitoring of such patients is generally conducted in two ways. One way is based upon symptoms. Recurrence of cancer is often, though not always, “announced” by the onset of symptoms, such as burning sensation during urination, visible blood in the urine, or, obstruction of the urinary system from tumor. However, a significant number of patients do not have any symptoms at all. A second way is asking the patients to urinate into a cup, and then healthcare providers (often including specialized doctors called cytopathologists) look at the urine under a microscope in order to look for cancer cells. This is reasonably accurate in most patients who still have their original bladders, though there is still a need to improve upon the sampling and diagnosis methods. However, in particular, patients who have had their bladders removed pose unique challenges. This is because their new bladders (typically made from a piece of their own intestine) usually contain a significant amount of mucus. The mucus comes from the fact that the intestine was used to make the new bladder, and the intestine thinks it is still “intestine”, so it does one of the things that all intestines do: it secretes mucus.

Normally, mucus secretion into urine from intestinal neobladders is not an issue for patients. But, for the cytopathologists who have to examine their urine under a microscope to look for cancer cells, it is a problem. The mucus “obfuscates” what they see in the microscope: instead of just plainly seeing the cells normally present in the urine, their field is shrouded by big clumps of mucus. These masses of mucus often contain within them the very cells of interest in the diagnosis. They will either be healthy normal sloughed urothelial cells, or, they will be sloughed malignant urothelial cells, when there is tumor present. A frustrated ability to recognize the difference, and the ability to find the samples for the determination in the first place, can have catastrophic consequences.

Accordingly, if a patient has had their bladder replaced with a new bladder made of intestine, conventional urine analysis is a relatively poor study to use to look for cancer cells. This is because the mucus makes it very hard to see the cells clearly. Even in the event that the mucus itself may not prevent a cytopathologist from seeing the cancer cells in a particular specimen, the mucus in the neobladder made of intestine acts like a “trap” for cells that slough-off the neobladder walls. Also, neobladders get inflammatory reactions frequently. The inflammatory state attracts macrophages (immune system cells), which also get trapped in the mucus. When the patient urinates, there is mucus inside the specimen. An additional problem is that the mucus is already loaded with cellular debris, and this debris is what prevents a good view of any urothelial cells that might be cancerous.

While the conventional diagnostic techniques using voided urine sample is still of some value, this is mainly because there are no real alternatives. Nonetheless, the “sensitivity” of such approach is low. A patient has to have a fair amount of tumor present before conventional techniques can detect it under the microscope. This is one suspected reason why patients with recurrent bladder cancer in the urethra tend not to survive for very long. By the time the recurrence is detected, the cancer has often already invaded the urethra, and spread into the blood and lymphatic systems (i.e. metastasized).

Notwithstanding these recognized shortcomings of current diagnostic techniques, the diagnostic quality of a urine specimen from a patient with a neobladder (e.g., made from the patient's own intestine) has not been appropriately studied or addressed in the past. Urologists have generally provided little challenge, study, or provocative discussion on the problem or possible solutions. This may be due, in one regard, to the fact that Cytopathologists, and not Urologists, are the ones who study the urine specimen. Moreover, the cytopathologists have also not conducted prolific study or debate about the poor quality of their specimens, possibly because it is intuitive to them—it is because of the mucus and cellular debris, etc., as a necessary evil of examining neobladder urine voids.

Accordingly, a significant need exists for providing improved systems and methods for collecting cellular material from urethral linings in order to efficiently, predictably, and accurately, diagnose medical conditions associated with the urethra. There is in particular a significant unmet need for improving the ability to diagnose cancer recurrence in patients with intestinal neobladders.

In addition to the foregoing shortcomings of the available systems and techniques for collecting and diagnosing cancer in cells along the urethral lining, a significant need also exists to improve the ability to locally treat cancer and its recurrence along the urethral lining in patients. To the extent chemotherapy agents may be delivered locally to the area, the correct combination of drug and delivery system has yet to be optimized for optimal results.

### SUMMARY OF THE INVENTION

One aspect of the invention is a medical device system that includes a transurethral catheter assembly with a proximal end portion and a distal end portion. An infusion lumen extends between a proximal infusion port along the proximal end portion and a distal infusion port positioned along the distal end portion adapted to infuse urethral wash medium into the urethra via the infusion lumen through the distal infusion port. The system includes means for collecting urethral wash contents created by infusing the urethral wash medium into the urethra through the distal infusion port.

According to one mode of this aspect the transurethral catheter assembly includes an inflatable distal balloon located on the distal end portion distally of the distal infusion port so as to isolate the urethral wash medium infused into the urethra through the distal infusion port to a region of the urethra proximally of the distal balloon and for proximal collection by the collection means.

According to one embodiment of this mode, the collection means comprises an aspiration lumen that extends between a proximal aspiration port along the proximal end portion and a distal aspiration port located along the distal end portion but proximally of the distal balloon. The urethral wash contents created by the urethral wash medium infused through the distal infusion port are aspirated through the distal aspiration port for collection.

According to one further embodiment of the embodiment just described, a second inflatable proximal balloon is provided along the distal end portion proximally of the distal balloon as a two-balloon system, with a longitudinal spacing therebetween configured to isolate a region of the urethra therebetween. The distal infusion port and distal aspiration port are both located between the two spaced balloons. This allows for flushing the isolated urethral region with the infused urethral wash medium and collecting the urethral wash contents through the distal aspiration port in a closed loop system between the balloons.

According to another mode, a cytology analysis system is provided in combination with the transurethral catheter assembly for conducting cancer diagnosis on the cells collected with the urethral wash contents.

According to another mode, a wash medium is coupled to the infusion lumen and comprises a material selected to substantially preserve cells collected in the aspirated urethral wash contents in a condition that is preferably well preserved for cytology evaluation in diagnosing cancer. The wash medium is not generally bioactive as to the collected cells nor generally therapeutic to the cells or the urethra, nor is it necessarily or required to be radiographically visible (i.e., radiopaque dye). In a particular exemplary embodiment of this mode, the flush medium comprises saline.

In another mode, a wash medium is coupled to the infusion lumen and comprises BCG for closed loop, isolated cancer therapy to the urethral region isolated between two spaced balloons.

In another mode, the infusion lumen extends to a distal port located distally of the infusion port and also distally of the distal balloon. A valve that comprises a deflectable member is located along a portion of an internal wall of the catheter assembly that separates the infusion lumen and an inflation lumen of the distal balloon. The valve is positioned distally from the distal infusion port and proximally of the distal balloon. The valve remains open relative to the infusion lumen when the distal balloon is deflated. This allows fluid communication between the distal port and a proximal port located proximally of the balloon assembly and outside the body, such as for example to facilitate drainage of urine from the bladder through the infusion lumen when the distal port is located within the bladder. The valve is actuatable to a closed condition relative to the infusion lumen as follows. Pressurization of the balloon inflation lumen during inflation of the distal balloon deflects the deflectable member into the infusion lumen to thereby collapse across and substantially close the infusion lumen to fluid communication between the distal port and the distal infusion port. This closed configuration allows for pressurized fluid within the infusion lumen to be expelled from the infusion lumen through the distal infusion port proximally of the inflated distal balloon.

In another mode, the two spaced balloon mode includes an adjustably spaced, telescoping arrangement between balloons, wherein an internal catheter member that includes the distal balloon telescopes distally from an outer catheter member that includes the proximal balloon. A coaxial space between the outer catheter member and inner catheter member may provide the infusion or aspiration lumen and port.

Another aspect is a medical device system that includes a transurethral catheter with two spaced balloons, and infusion and aspiration/collection lumens coupled to infusion and aspiration/collection ports located between the spaced balloons, respectively, on the distal end of the catheter.

According to one mode of this aspect, a volume of a substantially non-therapeutic, non-radiologically visible urethral wash medium. In one particularly beneficial embodiment, the wash medium is saline. In another further embodiment, a volume of saline plus urethral cellular contents and debris is coupled to the aspiration lumen. In another embodiment, the wash medium is coupled to the infusion lumen at substantially low pressure sufficient to provide for infused distal flow of the medium into the region and subsequent aspiration from the region, but below a pressure threshold sufficient to substantially dilate the region of urethra.

According to another mode, the system further comprises a diagnostic system configured to conducting a diagnostic procedure on cells collected with the urethral wash medium. In one embodiment, the diagnostic system is configured to be located externally from the patient while the transurethral catheter is located at least in part within the urethra. In another embodiment, the diagnostic system comprises a cytology diagnostic system. In a further embodiment, the cytology diagnostic system comprises a microscope.

According to another mode, the infusion lumen extends beyond the distal balloon to a distal port, and with a valve comprising a deflectable diaphragm located between the infusion lumen and the balloon inflation lumen and distally of the distal infusion port but proximally of the distal balloon, wherein the deflectable diaphragm of the valve is configured to deflect across and close off the infusion lumen upon pressurization of the balloon inflation lumen during balloon inflation.

According to another mode, the system further comprises a pressurizable volume of a urethral wash medium that comprises BCG chemotherapeutic agent coupled to the infusion lumen.

Another aspect of the invention is a method for collecting cells from a urethra for cytologic diagnosis, comprising: inflating two spaced balloons in the urethra to thereby isolate a region of urethral wall between the inflated balloons; while the balloons are inflated, infusing a volume of urethral wash medium into the region and aspirating a volume of urethral wash material from the region that includes the infused urethra wash medium and cells from the urethral wall; collecting the aspirated urethral wash material in a manner that preserves the wash material for cytological examination of the cells; and performing cytological diagnostic examination on the collected cells.

According to one mode, the diagnostic examination comprises diagnosing the cells for cancer.

Another aspect comprises a method for collecting cells from a urethra for cytologic diagnosis, comprising: inflating two spaced balloons in the urethra to thereby isolate a region of urethral wall between the inflated balloons; while the balloons are inflated, infusing a volume of substantially non-biologically active, non-therapeutic, non-radiopaque liquid into the region at a relatively low pressure insufficient to substantially dilate the region and aspirating a volume of urethral wash material from the region that includes the infused liquid and cells and debris from the urethral wall; and collecting the aspirated urethral wash material.

According to one mode of this aspect, the infused liquid comprises saline.

Another aspect of the invention is a method for treating cancer in a region of a urethra, comprising: inflating two spaced balloons in the urethra to thereby isolate a region of urethral wall between the inflated balloons; and

while the balloons are inflated, infusing a therapeutic dose of BCG to the region and aspirating the infused therapeutic dose of BCG from the region.

Another aspect of the invention is a method for performing a medical procedure in a urethra of a patient, comprising: transurethrally delivering a distal end portion of a catheter assembly at least in part to the bladder;

inflating a distal balloon on the distal end portion in the bladder; while the distal balloon is inflated in the bladder, infusing a volume of urethral wash medium into a region of urethra through a distal infusion port located proximally of the balloon on the distal end portion, and aspirating the volume of urethral wash medium from the region into an aspiration lumen of the catheter assembly through a distal aspiration port located proximally of the distal infusion port; and wherein the urethral wash medium comprises normal saline or BCG.

Another aspect of the invention is a method comprising: transurethrally delivering a distal end portion of a catheter assembly such that a distal balloon on the distal end portion is located within the bladder; while the distal balloon is deflated within the bladder, allowing fluid communication along a lumen between a distal port located distally of the distal balloon and a proximal port located along a proximal end portion of the catheter assembly located externally of the patient; inflating the distal balloon in the bladder by pressurizing a fluid located within a balloon inflation lumen coupled to the balloon; upon pressurization of the balloon inflation lumen, actuating a valve member to close the lumen between the distal port and an intermediate port located along the distal end portion proximally of the distal balloon; and wherein with the valve in the closed condition the proximal port fluidly communicates with the intermediate port via the lumen but not with the distal port.

Another aspect of the invention comprises inserting a balloon catheter transurethrally through a penis of a patient into the patient's bladder; inflating a distal balloon on the balloon catheter within the bladder; applying a clamp around the penis sufficient to squeeze the penis onto the catheter; infusing a liquid into the urethra through an infusion port along the catheter between the clamp and the distal balloon; and aspirating the infused liquid from the region.

Another aspect of the present invention is a medical device system that includes a transurethral catheter assembly with a proximal end portion and a distal end portion configured to be positioned at least in part along a urethra in a patient with the proximal end portion extending externally of the patient's body. An infusion lumen extends between a proximal infusion port along the proximal end portion and a distal infusion port positioned along the distal end portion. The infusion lumen is adapted to infuse a wash medium into the urethra via the infusion lumen through the distal infusion port. A collection system is configured to collect urethral wash contents created by infusing the wash medium into the urethra through the distal infusion port.

Another aspect is a medical device system with a transurethral catheter assembly with a proximal end portion and a distal end portion that is configured to be positioned at least in part within a urethra of a patient. A distal expandable member is located along the distal end portion, whereas a proximal expandable member located along the distal end portion proximally of the distal expandable member. The proximal and distal expandable members are longitudinally separated from each other along the distal end portion. An infusion lumen is fluidly coupled to a proximal infusion port located along the proximal end portion and a distal infusion port located along the distal end portion between the two spaced expandable members. A collection system is also included that comprises a collection lumen fluidly coupled to a proximal collection port along the proximal end portion and a distal collection port located along the distal end portion between the two spaced expandable members.

Another aspect is a medical device system that includes a transurethral catheter assembly with a proximal end portion and a distal end portion that is configured to be positioned at least in part within a urethra of a patient. A luminal isolation assembly is located along the distal end portion and that is configured to substantially isolate at least a region of the urethra for local fluid delivery. A volume of wash medium is coupled to the catheter in a manner configured to selectively infuse the volume into the region of urethra isolated by the luminal isolation assembly. A collection system is configured to collect urethral wash contents created by infusing the urethral wash medium into the region of urethra isolated by the luminal isolation assembly.

Another aspect of the present invention is a medical device system that includes a catheter assembly with a proximal end portion and a distal end portion that is configured to be positioned at least in part within a lumen of a patient. A luminal isolation assembly is located along the distal end portion and that is configured to substantially isolate at least a region of the lumen for local fluid delivery. An infusion lumen is fluidly coupled to a proximal infusion port located along the proximal end portion and a distal infusion port located along the distal end portion so as to infuse fluid to the region of the lumen isolated by the luminal isolation assembly. A collection system is provided that includes a collection lumen fluidly coupled to a proximal collection port along the proximal end portion and a distal collection port located along the distal end portion so as to collect material washed from the region of the lumen isolated by the luminal isolation assembly. A volume of wash medium is located within the infusion lumen. A volume of flushed material is located within the collection lumen that comprises cells suspended within the wash medium.

According to one mode, the wash medium is substantially non-therapeutic. In another mode, the wash medium is substantially non-radiographically visible. In another mode the wash medium is adapted to substantially preserve the cells for cytology analysis and cancer diagnosis.

Another aspect is a medical device system with a catheter assembly with a proximal end portion and a distal end portion that is configured to be positioned at least in part within a lumen of a patient. A luminal isolation assembly is located along the distal end portion and that is configured to substantially isolate at least a region of the lumen for local fluid delivery. An infusion lumen is fluidly coupled to a proximal infusion port located along the proximal end portion and a distal infusion port located along the distal end portion so as to infuse fluid to the region of the lumen isolated by the luminal isolation assembly. A collection lumen is fluidly coupled to a proximal collection port along the proximal end portion and a distal collection port located along the distal end portion so as to collect material infused into the region of the lumen isolated by the luminal isolation assembly. A volume of wash medium is coupled to the infusion lumen for infusion into the region of lumen isolated by the luminal isolation assembly. The wash medium comprises BCG.

According to one further mode of the aspects, a radially expandable distal member is provided and is located on the distal end portion distally of the distal infusion port. The distal infusion port is located proximally of the distal member. The expandable distal member when expanded is configured to isolate the wash medium infused into the urethra through the distal infusion port to a region of the urethra located proximally of the distal member. The collection system is configured to collect the isolated infusion of the wash medium.

According to another mode, an infusion lumen extends between a proximal infusion port along the proximal end portion and a distal infusion port positioned along the distal end portion proximally of a distal expandable member. The infusion lumen is adapted to infuse the volume of wash medium into the isolated region of urethra through the distal infusion port;

In further embodiments of aspects and modes incorporating expandable members, the member is an inflatable balloon. In one further embodiment, a distal port is located distally of a distal infusion port and also distally of a distal balloon. The infusion lumen is coupled to the distal port, and an adjustable valve is coupled to the infusion lumen and is adjustable between an open condition and a closed condition. In the open condition the proximal infusion port communicates via the infusion lumen with the distal port. In the closed condition the proximal infusion port communicates via the infusion lumen with the distal infusion port but not with the distal port.

According to one further embodiment, the valve comprises a deflectable member located along a portion of an internal wall of the catheter assembly that separates the infusion lumen and an inflation lumen of the distal balloon. In further variations, the deflectable member is positioned distally from the distal inflation port, and the valve remains open relative to the infusion lumen when the distal balloon is deflated, thereby allowing fluid communication between the distal port and the proximal infusion port. The valve is actuatable to a closed condition relative to the infusion lumen upon pressurization of the balloon inflation lumen during inflation of the distal balloon, thereby deflecting the deflectable member into the infusion lumen to collapse across and substantially close the infusion lumen with respect to fluid communication between the distal port and the distal infusion port. The valve in the closed configuration allows for pressurized fluid within the infusion lumen to be expelled from the infusion lumen substantially through the distal infusion port proximally of the inflated distal balloon.

According to another mode of the various aspects, the collection system comprises a collection lumen that extends between a proximal collection port along the proximal end portion and a distal collection port located along the distal end portion proximally of the distal member. The injected or irrigated wash contents created by the wash medium infused through the distal infusion port are collected through the collection lumen via the distal collection port.

According to another mode, proximal and distal expandable members are provided on the catheter and comprise inflatable proximal and distal balloons, respectively.

In one beneficial embodiment, an outer catheter member with a proximal end portion and a distal end portion and an inner lumen cooperates with an internal catheter member with a proximal end portion and a distal end portion and that is located at least in part within the inner lumen of the outer catheter member and with the distal end portion of the internal catheter member extending distally from the inner lumen. The proximal balloon is located along the distal end portion of the outer catheter member. The distal balloon is located long the distal end portion of the inner catheter member. The inner catheter member is slideable longitudinally relative to the outer catheter member in a telescoping arrangement such that the spacing between the proximal balloon and distal balloon is adjustable.

According to one further feature of this embodiment, a coaxial space is located between the outer catheter member and inner catheter member, and at least one of the infusion lumen or collection system is fluidly coupled to the coaxial space.

According to still another mode, the system includes a cytology analysis system configured for conducting cancer diagnosis on cells collected via the collection system, such as a microscope.

In another mode, a wash medium is used in the system for isolated luminal flushing and comprises a material that substantially preserves cells collected with the wash medium for cytology evaluation in diagnosing cancer. In another mode the wash medium is not substantially bioactive as to cells collected with the wash medium. In another, it is not substantially therapeutic to the lumen. In another, it is not substantially radiographically visible. In another mode, it is saline.

In yet another mode, the wash medium includes BCG and the system is therapeutic.

In another mode, the catheter comprises an elongated body constructed of a substantially flexible elastomer.

In further modes, a removable stylet or guidewire may be used for delivery. In another mode, the balloon expands under volumetric control.

In another mode, an adjustable penile clamp is also provided in the system and is configured to be positioned externally around a portion of a penis and to clamp the portion around the catheter when the distal end portion of the catheter is located within a lumen, such as a urethra, and in a manner that substantially isolates a region of the urethra distally from the clamped portion from communicating outside the body through the urethra proximally of the clamp.

Another aspect is a method for collecting cells from a urethra for cytologic evaluation and cancer diagnosis. It includes solating a region of urethra for local, isolated delivery of a wash medium; infusing a volume of wash medium into the isolated region; and collecting a volume of flushed material that comprises cells flushed from the isolated region with the infused volume of wash medium in a manner that preserves the cells in a condition sufficient to conduct cytological examination of the cells for cancer diagnosis.

Another aspect is a method for collecting cells from a urethra for cytologic diagnosis, comprising: inflating two spaced balloons in the urethra to thereby isolate a region of urethral wall between the inflated balloons; while the balloons are inflated, infusing a volume of substantially non-biologically active, non-therapeutic, non-radiopaque wash medium into the region; collecting a volume of urethral wash material that includes cells together with the infused wash medium from the region.

Another aspect is a method for treating cancer in a region of a lumen in a urinary tract in a patient, comprising: inflating two spaced balloons in the lumen to thereby isolate a region of lumenal wall between the inflated balloons; and while the balloons are inflated, infusing a therapeutic dose of BCG to the region and aspirating the infused therapeutic dose of BCG from the region.

Another aspect is a method for performing a medical procedure in a lumen of a urinary tract in a patient, comprising: transurethrally delivering a distal end portion of a catheter assembly at least in part to the bladder; infusing a volume of wash medium from the distal end portion into a region of the lumen around the catheter; preventing the infused wash medium from flowing distally and proximally from the region; and collecting the infused wash medium from the region via a distal collection port located along the catheter in the region.

Another aspect is a method for controlling fluid communication along a catheter in a urinary tract of a patient, comprising: positioning a distal end portion of the catheter transurethrally along the urinary tract and with a proximal end portion of the catheter located externally of the patient; and adjusting an internal valve coupled to an internal lumen of the catheter from an open condition to a closed condition. In the open condition the lumen communicates between a proximal port along a proximal end portion, an intermediate port along the distal end portion, and a distal port along the distal end portion distally from the intermediate port. In the closed condition the lumen is closed between the proximal port and the distal port, but open between the proximal port and the intermediate port.

Another aspect is a method for performing a medical procedure in a urinary tract of a patient, comprising: translumenally delivering a distal end portion of a catheter assembly such that a distal balloon on the distal end portion is located at least in part within the bladder; providing fluid communication along a lumen between a proximal port located along the proximal end portion externally outside of the patient and a distal port located along the distal end portion; inflating a balloon located along the distal end portion proximally of the distal port via delivery of a pressurized fluid through an inflation lumen into the balloon; and closing the lumen between the distal port and the proximal port at least in part by inflating the balloon. While the lumen is closed between the distal port and the proximal port, a wash medium is infused through an intermediate port located proximally of the inflated balloon and via the proximal port.

Another aspect is a method for treating cancer in a patient, comprising: inserting a catheter into a lumen of a urethra or ureter of a patient; isolating a region of the lumen from proximal or distal flow around the catheter; infusing BCG into the region; collecting the BCG from the region.

Another aspect is method for performing a medical procedure relative to a patient, comprising: inserting a balloon catheter transurethrally through a penis of a patient into the patient's bladder; inflating a distal balloon on the balloon catheter within the bladder; applying a clamp around the penis sufficient to squeeze the penis onto the catheter; and infusing a liquid into the urethra through an infusion port along the catheter between the clamp and the distal balloon; and aspirating the infused liquid from the region.

The systems and methods herein summarized may be provided together, or in separate component parts or steps and still provide significant value as further contemplated aspects of this disclosure. In addition, the various systems and related components described may be chosen from a kit of various sizes and specific embodiments in order to suit a particular medical need or patient anatomy, as would be apparent to one of ordinary skill based upon review of the totality of this disclosure. In addition, medical systems are often provided in sterile, packaged form, with packaging inserts describing the instructed methods for their use. These aspects are also considered further aspects of additional value, both independently and in combination with the various other aspects and modes described.

The invention further contemplates additional combinations and sub-combinations of the various aspects, modes, embodiments, features, and variations herein shown and described as would be apparent to one of ordinary skill in the art based at least in part upon this disclosure.

### BRIEF DESCRIPTION OF THE SEVERAL VIEWS OF THE DRAWINGS

The invention will be more fully understood by reference to the following drawings which are for illustrative purposes only:

FIG. 1 shows a plan view of one type of balloon catheter used in the urinary tract.

FIG. 2A shows a plan view of another catheter constructed by modifying a catheter like that shown in FIG. 1.

FIG. 2B shows a transverse cross-sectional view taken along line 2B-2B shown in FIG. 2A.

FIG. 3A is a plan view of the same catheter shown in FIG. 2A except in a different circumferentially rotated orientation.

FIG. 3B shows a transverse cross-sectional view taken along line 3B-3B shown in FIG. 3A.

FIG. 4A shows a picture of an elevational view of two catheters, one of a conventional type such as shown in FIG. 1 (above), the other a similar catheter, but modified according to FIGS. 2A-3C.

FIG. 4B shows an exploded view of the distal end portion of the catheters shown in FIG. 4A.

FIG. 4C shows a still further exploded view of the picture shown in FIG. 4B and showing the finer detail of the distal end portion of one of the conventional catheter.

FIG. 4D shows a similar view to FIG. 4C except for the modified catheter.

FIG. 5 shows a photograph of an elevational view of a modified catheter such as that shown in FIGS. 4A-B and including certain additional cooperating components of an overall system.

FIG. 6 shows another catheter embodiment, similar to the modified catheter shown in FIGS. 2A-5 during transurethral use in a patient shown in shadow.

FIG. 7A-B show angular perspective view photographs of a penis clamp embodiment during two modes of use, respectively.

FIG. 8 shows another catheter embodiment during one mode of use.

FIG. 9 shows one additional proximal assembly for use with the embodiment similar to that shown in FIG. 8.

FIG. 10A shows a plan view of another catheter embodiment.

FIG. 10B shows a transverse cross-sectional view taken along lines 10B-10B in FIG. 10A.

FIG. 11A shows a plan view of the same catheter shown in FIG. 10A embodiment except with the balloon inflated.

FIG. 11B shows a transverse cross-sectional view taken along lines 11B-11B in FIG. 11A.

FIG. 12 shows a microscope magnified view of certain cells of a voided urine sample from a patient with a neobladder, and shows certain particular types of cells.

FIG. 13 shows a microscope magnified view of urethral wash content taken according to certain embodiments of the present disclosure.

### DETAILED DESCRIPTION OF THE INVENTION

Various aspects, modes, and embodiments of the present invention are further described and variously illustrated as follows.

By convention, the end furthest inside the patient (furthest from the outer skin surface) has been herein designated the “distal end”, while the “proximal end” of the catheter is the end that remains either outside of the body, or, the working end of the catheter, through-which fluids are irrigated into/out of the catheter. Medical conventions typically refer to proximal as “upstream” and distal as “downstream” when referring to anatomy, such as down the urological tract from “proximal” structures such as the kidney or ureters and “distally” toward the outside world such as via a urethra. For consistency of this disclosure, o the extent catheters devices and methods are herein described in retrograde delivery within such anatomies, “proximal” will hereunder mean outward from the patient and “distal” will hereunder mean further inward or upstream into the patient, including with respect to such contextual description of anatomy associated with the device intervention.

One highly beneficial aspect of the present disclosure provides a system and method that isolates the urethra from the bladder, so that only the urethra can be and is perfused via the catheter, such that the fluid delivered through the catheter cannot enter the bladder (i.e. a catheter is configured to be blind-ending at the tip). By isolating the urethra for perfusion, several types of fluids can be introduced. In various highly beneficial embodiments of the present disclosure provide such local delivery of certain drugs for novel and highly beneficial cancer therapy applications, and/or irrigants for novel and highly beneficial diagnostic purposes. For example, irrigants so delivered in certain particular embodiments include generally non-bioactive and non-therapeutic wash irrigants, such as for example saline, followed by collection wherein the wash contents are well represented and preserved in the collection for improved analysis and diagnostic capabilities. Such local delivery of particular drug may be as appropriately determined for a specific “treatment” specified in a particular application, whereas the present disclosure includes a particular exemplary embodiment considered highly beneficial in particular for treating cancer in the urethra.

Various prior catheter-based delivery systems previously described in relation to transurethral use generally feature the delivery of therapeutic drugs, x-ray diagnostic agents (dyes), or other specified liquids, all generally intended for achieving certain in-vivo results from the infused material. However, various of the present embodiments instead provide a system and method for local irrigation and isolated collection of urethral wash materials for external diagnosis, such as according to various present aspects, modes, and embodiments of the invention, as has not been heretofore described. Such embodiments include incorporation into the delivery system and method of an irrigating wash agent that is substantially non-biologically active, and non-therapeutic with respect to the urethral region. X-ray visibility is not required of the liquid in many applications contemplated hereunder. Accordingly, omission of such radiopaque dye materials from the irrigant may in many cases provide a simpler solution and more robust for the other intended results of cell collection, preservation, identification, and diagnosis. The irrigating wash and collection may be performed at significantly low pressures merely required to be sufficient to collect the urothelial cells for external examination. In one particular and highly beneficial exemplary embodiment, such system and method relies solely on the incorporation of saline within the system and for washing and collecting the samples.

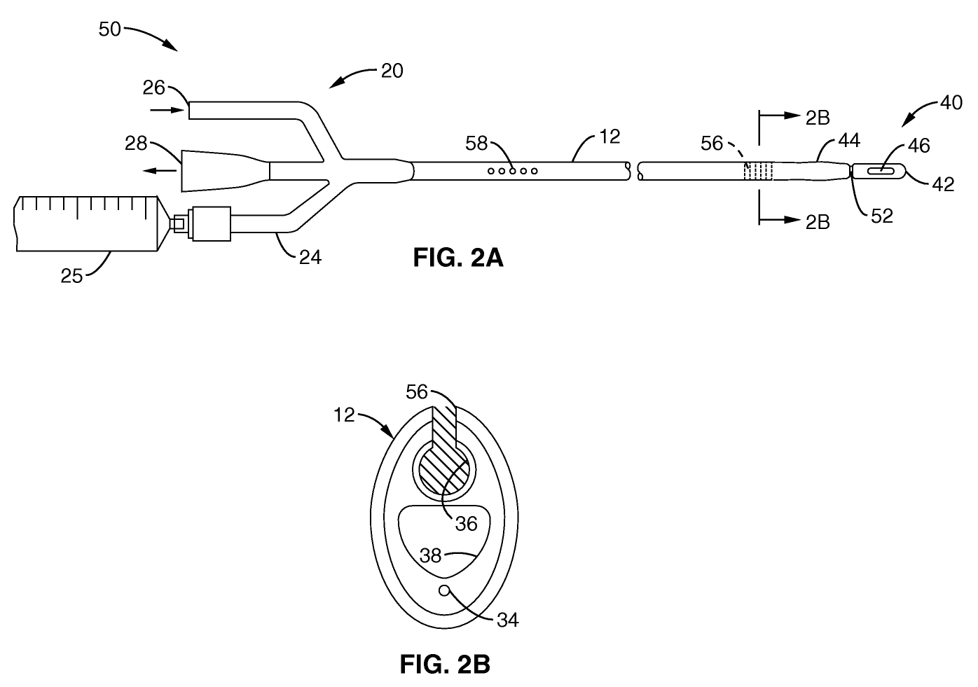

Certain particularly beneficial aspects of the present disclosure are herein described by reference to one particular embodiment variously shown and herein described by reference to FIGS. 2A to 6, and is further described by reference to certain human clinical use experiments performed as described further below.

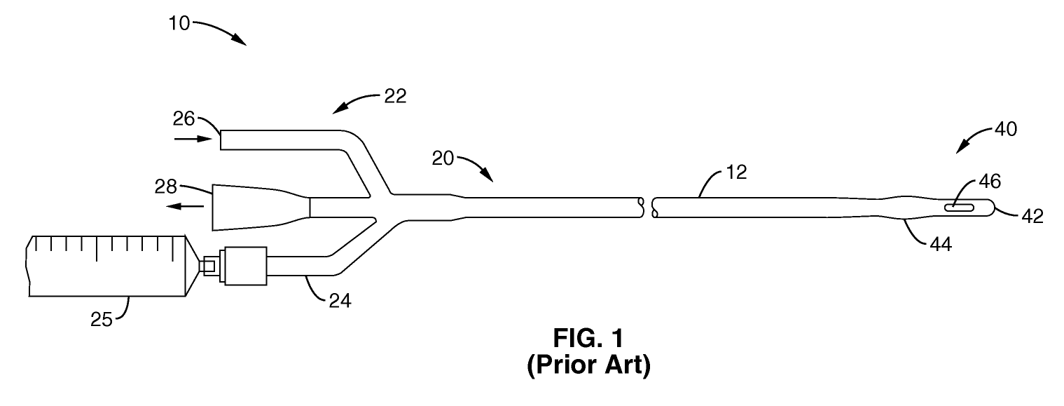

FIG. 1 shows a catheter (10) similar to a typical “Foley” type of catheter, which is commercially available. Catheter (10) includes an elongated body (12) extending between a proximal end portion (20) and a distal end portion (40). Distal end portion (40) includes a distal expandable member, shown as one particularly beneficial embodiment in FIG. 1 as inflatable balloon (44), that is located proximally adjacent to distal tip (42). A distal port (46) is located distally of balloon (44). When balloon (44) is inflated and seated at the urethral junction with the bladder, the bladder is thus isolated from the urethra such that fluid may be infused into or collected from the bladder via distal port (46) in a manner isolated from the urethra. In addition, in typical assemblies of this “Foley” type, a second distal port (not shown) is also provided for either of infusion or collection whereas the first distal port (46) provides the other of these functions.

Proximal end portion (20) includes a hub assembly (22) with a balloon inflation port (24) shown coupled to a removable syringe (25). The syringe (25) is typically provided separately and coupled for example via a conventional friction fit or luer lock coupling. Balloon inflation port (24) is coupled to balloon (44) via an inflation lumen extending therebetween in the elongate body (12)(not shown). Hub assembly (22) also includes a proximal infusion port (26) and a proximal collection port (28), which are coupled via separate infusion and collection lumens (not shown) along elongate body (12). These proximal ports are provided for inflow and outflow of fluid for expulsion and collection, respectively, from the catheter (1) distal to balloon (44), such as described above via distal port (46) and a second distal port as is most typically also provided.

It will be appreciated from the present description that certain of the present embodiments offer selective, closed-system irrigation/perfusion of a closed space or open-ended tube structure within the body. Certain more specific embodiments are comprised of a single to multi-lumen catheter with clusters of hole-perforations along the side(s) of the catheter, which each open into at least one of the separate longitudinally oriented lumens connecting one end of the catheter to the other. One or both of the ends of the catheter can be inflated to a balloon shape, whereby the balloon serves to occlude the passage of fluid along the lumen being irrigated/perfused.

The present embodiments differ from the traditional “Three-way Foley catheter”, though such platform can be and has been modified according to certain ones of the present embodiments to accomplish certain objectives of stated in the present disclosure. This other conventional catheter is called a “three-way” because its tubular structure contains three separate dedicated channel lumens. At the proximal end, each channel flares into a hub-port. Distally, one of the three channels ends as a balloon which, when not filled, lies flat. When filled, the balloon is a spherical shape, and made of latex or silicone, as is the rest of the catheter. The remaining two of three channels open to the outside through a hole at the tip of the catheter (located between the balloon and the “eye” opening, such as shown at the tip as port (46) in FIG. 1.

The conventional “3-way catheter” is generally used after limited resection of the prostate gland in males. The prostate lies between, and connects, the bladder and the urethra. After resection of prostate tissue, a catheter must be used to drain the bladder, as the patient will not be able to urinate on his own. However, there is nearly always a lot of blood at the operative site, and this blood will clot, and obstruct the catheter inlet-hole. Therefore, a special catheter with an extra channel (the 3-way Foley catheter) is used. In all, the 3 channels are used for the following: one is dedicated to fill/empty the balloon end; the second is used to drain the bladder, and the third is used to instill water into the bladder. The purpose of the balloon is to prevent the catheter from slipping out of the bladder.

Further aspects of typical use given to catheter (10) shown in FIG. 1 are described as follows for purpose of illustration. After placing the catheter within the urethra, the balloon is filled with 10-15 cc sterile water (by introducing the water through the corresponding sealed hub-port at the proximal end of the catheter, e.g., port (24) in FIG. 1. Next, the chosen irrigant fluid is instilled into the catheter through the hub-port designated as port (26). The irrigant instilled through the infusion channel coupled to port (26) drains out of the catheter at the tip, and fills the bladder, washing dissolving any forming blood clots. The irrigant (and any urine produced by the bladder) drain out of the bladder through the channel connected to the other proximal collection port such as that port (28) shown in FIG. 1

In contrast, one particular embodiment of the present disclosure provides a catheter (See FIG. 2A) designed to allow irrigant to fill and bathe the length of space between the balloon and the proximal-most end of the catheter, for example the urethra between the balloon and the penis tip.

More specifically, FIG. 2A shows catheter (50) representing one beneficial current embodiment of the present disclosure, as constructed via certain modifications made to the prior art catheter (10) shown in FIG. 1. Similar structures as shown and described by reference to catheter (10) in FIG. 1 are given similar reference numerals here to such similar structures provided in catheter (50) in FIG. 2A. However, in FIG. 2A three significant modifications are made as shown, and described as follows.

According to one such modification, a constriction (52) is made to the distal end portion (40) distally of balloon (44) and proximally of distal port (46) such that fluid communication with distal port (46) is occluded within elongate body (12) proximally of constriction (52).

As a second modification, distal infusion ports (56)(shown in shadow in FIG. 2A as running transverse to the view, and in cross-section in FIG. 2B) are formed at a location proximally adjacent to balloon (44) as holes or apertures through elongate body (12) and into infusion lumen (36) (FIG. 2B). Infusion lumen (36) otherwise couples proximal infusion port (26) with distal port (46) but for the modification providing the occlusive constriction (52) of this embodiment. Accordingly, fluid being injected through proximal infusion port (26) is delivered along infusion lumen (36) and out of elongate body (12) through distal infusion port (56) proximally of balloon (44). This contrasts to infusing the fluid via that same infusion lumen (36) distally of balloon (44) as would be the case without these two modifications providing constriction (52) and distal infusion ports (56).

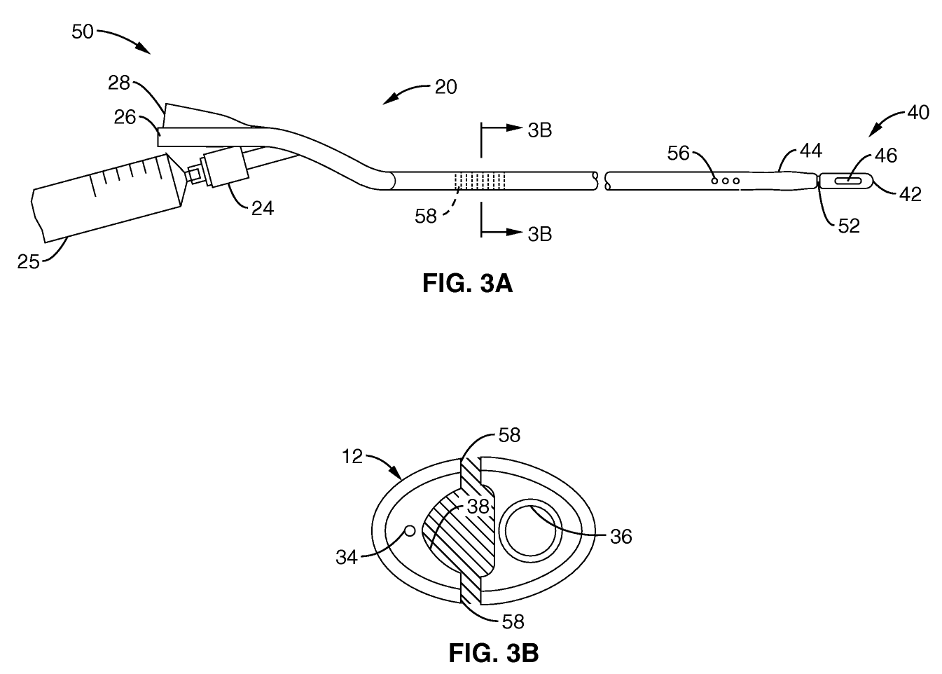

As a third modification, distal collection ports (58) are formed along the distal end portion (40) proximally of distal infusion ports (56), also as holes or apertures through elongate body (12). However, as shown in FIG. 3B, these holes are formed into collection lumen (38), which in the particular embodiment shown may be formed through multiple sides or circumferential positions around elongate body (12) to enhance collection. Collection lumen (38) is also prevented from communicating outside of elongate body (12) distally of balloon (44) due to further occlusive effect of constriction (52). Hence, fluid communication in and out of elongated body (12) via this collection lumen (38) is restricted to flow between proximal collection port (28) and distal collection port (58).

Other features of the catheter embodiments herein shown in the Figures, such as may be reflected for example in cross-sectional views of catheter wall features shown for purpose of illustration in FIGS. 2B and 3B, respectively, are illustrative only and not considered essential unless so specifically stated. While certain such constructions shown may be considered particularly beneficial, they are otherwise considered illustrative examples of broader aspects, and other modifications as to such specific structures or materials may be made suitable for many applications to the extent apparent to one of ordinary skill.

The following provide additional description of the related features shown in FIGS. 2A-3B for purpose of providing further understanding. It is to be appreciated that the respective balloon (44) is not shown inflated in these Figures for clarity, but is inflatable to an expanded condition. Moreover, it is also to be appreciated that he distal end of the present catheter (50) is closed blind-ending. In FIG. 2A, the tip (42) of the catheter (50) is shown sutured-closed. Thus the present catheter (50) differs from the conventional 3-way Foley catheter shown as catheter (10) in FIG. 1, which is designed to irrigate distal to the balloon (e.g., into the bladder). To achieve this result for the present embodiments, the irrigant does not drain out of the tip of the catheter, but proximal to the balloon, through the hole-perforations such as those shown at ports (56) in FIG. 2A and FIG. 2B. The cross-sectional view of the irrigant hole-perforations is shown in FIG. 2B.

The irrigant is instilled through port (26). Because all of the lumens are closed past the point of where the balloon is located, the irrigant is forced to exit via the hole-perforations at port (56). If the balloon is filled, and is occluding the distal end of the urethra (the bladder neck), then the irrigant instilled through port (26) is forced to pass along the outside of the catheter, proximally towards the tip of the penis. To collect the irrigant as it approaches the distal end of the penis, holes are made along the wall of the proximal end of the catheter. These holes open only into the collection channel or lumen (38) as shown in FIG. 2B. Collection lumen (38) terminates at hub-port (28), the drainage (outflow) port.

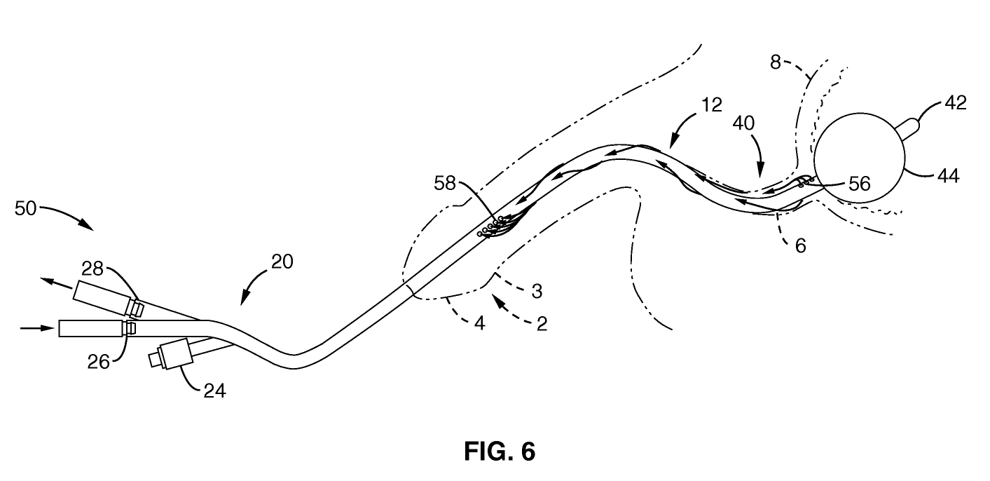

As further shown in FIG. 6, a drainage tube can be inserted into hub-port (28), to collect the effluent irrigation fluid in a sterile (if desired) closed-system fashion. FIG. 6 shows a schematic representation of the distal end of such a drainage tube inserted in hub-port (28), and a schematic representation for irrigant (inflow) tubing in hub-port (26). FIG. 6 also shows the administration of an irrigant to a male patient: the irrigant enters hub-port (26) (inlet port), exits distally through hole-perforations (56), is forced to drain towards the proximal end of the catheter due to the presence of the balloon (44), bathes the urethra (6) as it migrates towards the proximal end of the catheter, and drains back into the catheter through hole-perforations of port (58) corresponding to channel collection lumen (38) and located along the side of the catheter. Once the fluid has drained back into the catheter, it exists through hub-port (28) into an awaiting drainage tube as shown.

If the irrigant is a chemical drug that is hazardous to handle, it is quite beneficial to have the irrigant drain back into the catheter through the hole-perforations of collection ports along the catheter as described, for closed-system drainage, as this minimizes risk of surface contact for the patient or the physician delivering the chemotherapeutic agent.

Though considered particularly beneficial, use of the present device is not necessarily limited to the design, method or anatomic location described above in all circumstances. For instance, the proposed device may contain any number of channel lumens: a minimum of 2, to any maximum number, in order to meet a particular need.

Also, while particular embodiments shown and described are considered of particular benefit, in other modifications to meet particular needs the present device may have more anywhere from zero to more than 1 balloon, located at anywhere along the shaft of the catheter.

Furthermore, to obviate the need for extra lumens within the shaft of the catheter, to accommodate balloon(s), the balloon unit can also take the form of a flat “cuff”, lying on the surface of the catheter shaft, which is connected to a fine tube hub-port, through which the “balloon-cuff” is filled/emptied (illustration not shown).

It is contemplated that the catheter tip need not necessarily be exactly as illustrated. For example, the catheter tip may be curved (as desired), similar to a “Coude” tip catheter. The distal tip (just distal to the balloon) may be longer or shorter, as desired. Furthermore, the device may me blind-ending (as described above), or, reversibly “blind/open” ended, such as for example as provided by further embodiments elsewhere herein described. Furthermore, the catheter system can be modified to different thicknesses or constructions as may be appropriate for a particular application, for use within larger structures, such as the urethra, bowel, bladder, etc., to much narrower/finer structures, such as blood vessels, the upper urinary tract (ureter and kidney).

One particular novel and beneficial embodiment provides perforations along the shaft of the catheter, with flow isolated by one or more balloons. This allows selective perfusion: the option to perfuse only the space proximal to a distally located balloon, distal to a proximally located balloon, or in-between two balloons located anywhere along the catheter. Furthermore, such selectively located hole-perforations allow for “closed-system” irrigation, if desired. Closed system irrigation affords certain benefits as well, such as:

1. The irrigation pressure can be maintained high (or low, as desired), without risk of detriment to neighboring tissues, lying outside of the perfusion field, which would otherwise be harmed by high irrigation pressures. And/or prolonged exposure to the irrigant.

2. A “closed-system” is especially useful when the fluid used as the irrigant is a contact and/or exposure hazard, such as many chemotherapeutic agents. In such cases, it would be hazardous to allow the irrigant to simply “dribble out of” the perfusion field, to be collected manually/by gravity, etc.

Ideally, the location of any hazardous chemical agent should the controlled and secured at all times, while minimizing unwanted contact/exposure to the patient/health-care giver, etc. The present perfusion system allows the irrigant to be collected into a closed tube/bag system from within the patient, thereby minimizing hazardous contact.

3. If the specific irrigant can be re-administered to the same patient during the same treatment, then, to save cost, the outflow irrigant collected from the collection hub-port can be re-instilled (directly or in-directly) through an infusion hub-port to complete a circuit. Re-cycling the irrigant would likely not be an option in an open collection system, as the sterility of the irrigant would be lost once it was exposed to the outside environment.

Among other contemplated benefits, the present embodiments may be manufactured for use in select patients, such as those with certain intraurethral tumors for purposes of treatment and/or surveillance with intraurethral washings.

Again, while of particular benefit, the catheter system is not necessarily limited to the genitourinary system, and it can be appropriately modified as to structure and/or use as appropriate to perfuse any luminal closed-space structure within the body.

The ability to selectively perfuse the urethra (or any lumen space within the body) in a closed-system fashion as herein described by the particular device embodiments, or particular methods, and various combinations thereof, is considered of particular benefit.

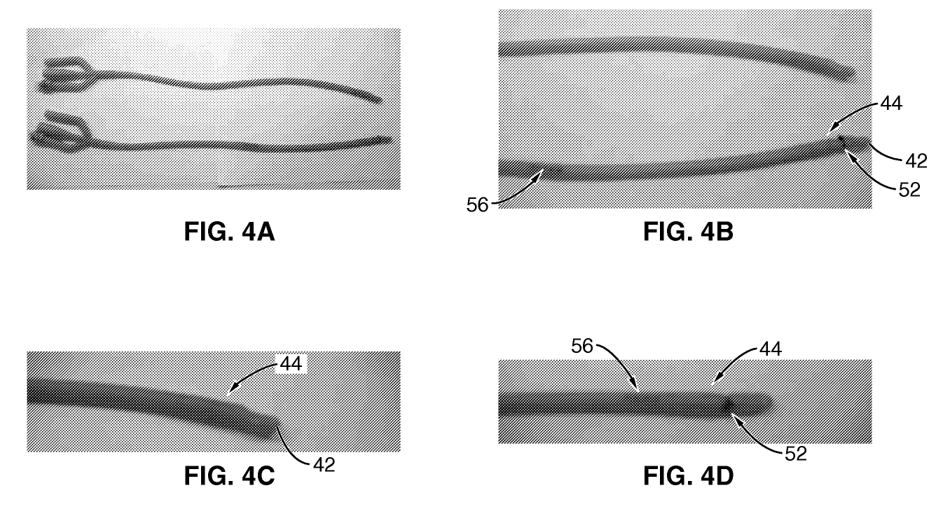

For purpose of providing a complete understanding of certain of the present embodiments, FIGS. 4A-D show photographs variously comparing a conventional “Foley” type catheter with one modified according to the present embodiments shown and described by reference to FIGS. 2A-3B.

More specifically, FIG. 4A shows a photograph of an entire conventional “Foley” type catheter unmodified (above) and a similar catheter as modified according to the embodiments of FIGS. 2A-3B (below).

FIG. 4B shows a higher magnification view of the same picture shown in FIG. 4A. This reveals in particular certain salient features of the distal end portion of the modified version of the present embodiment (below). Namely, these include: (a) a constriction (52) formed using a suture material tied tightly down around the catheter between the distal tip (42) and an inflatable balloon section (44); and (b) distal collection ports (58) that include a longitudinally spaced array of holes through the catheter located proximally down the catheter from the balloon (44).

FIG. 4C shows still a higher magnification view of the same picture shown in FIGS. 4A-B, but showing only the most distal region of the conventional un-modified catheter where its tip (42) and expandable region where balloon (44) are located. FIG. 4C is provided for comparison with the similarly magnified view of FIG. 4D, provided that FIG. 4D instead shows the higher magnification view of the modified catheter of the present embodiments. This further shows the sutured constriction (52) adjacent to the region of balloon (44), and distal infusion ports (56) are now shown in this orientation (vs. the other orientation shown in FIG. 4B that instead showed the distal collection ports (58) that are formed at a different circumferential location around the modified catheter).

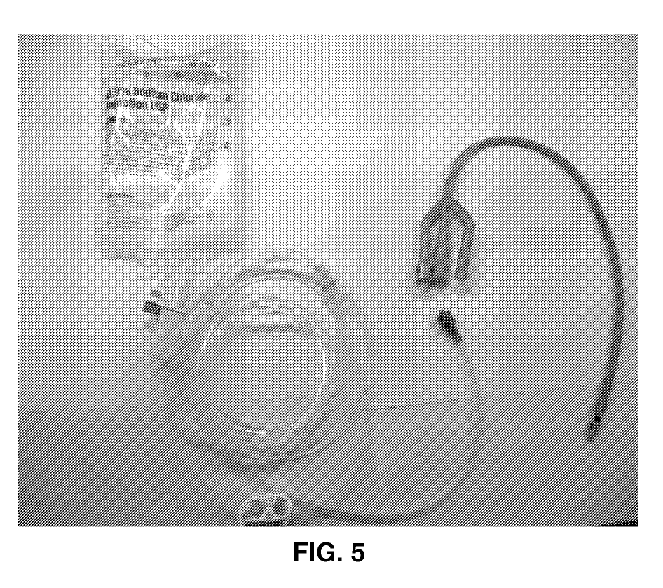

For still further illustration, FIG. 5 also shows a picture of the overall “modified” catheter of the present embodiment shown in FIGS. 4A-D in overall context in combination with a saline infusion/drip bag as a source for saline infusion to couple with the proximal infusion port of the catheter. This assembly is beneficial for ultimate use in selective urethral infusion (and as may include ultimate collection) of saline as herein contemplated in certain further embodiments to be further developed below. As is elsewhere developed hereunder, such source may be only the saline itself (such as for collection of urethral wash and cancer diagnosis), or may be saline or other fluid that containing a therapeutic compound for local therapy.

One particular beneficial use of the present embodiments, such as for catheter (50) shown and described above, is illustrated in FIG. 6 as follows. This illustration is shown by reference to a male patient's penis (2), and respective urethra (6) and bladder (8), whereas it is clearly contemplated that the devices and uses of the present embodiments may be applied similarly to female anatomy and uses, including as may be modified by one of ordinary skill where appropriate to more suitably adapt to anatomical considerations between genders.

According to the use represented in FIG. 6, distal end portion (40) of catheter (50) is inserted into the patients' penis (2) via the urethra (6) and until balloon (44) is located within bladder (8) just upstream of the junction between bladder (8) and urethra (6). Proximal end portion (20) remains outside of the penis (2). For diagnostic applications with the objective to collect cells that are well preserved in the irrigant for cytology and cancer diagnosis, one particular further embodiment lubricates the catheter outer skin using only water or saline—lubricant gels otherwise typically used for transurethral applications for comfort reasons may adversely affect the intended condition of the collected wash contents for evaluation. Once placed as shown, the balloon is inflated, such as by filling it with between about 10-20 ml of water, and the inflated catheter is then placed to tension. This tightens the seal the balloon is making at the bladder neck or junction. At this point, fluid delivered into proximal infusion port (26) is infused out from elongated body (12) through distal infusion port (56) into urethra (6). Balloon (44) isolates this infusion to the urethra (6) by blocking distal communication up into bladder (8). Furthermore, the injected fluid in the urethra (6) is then collected through distal collection ports (58) and ultimately through proximal collection ports (28), which may be coupled to a container or discarded, depending upon the particular application for the isolated urethral irrigation and collection.

In one particular beneficial further embodiment and application, saline is coupled to infusion lumen (36) of the catheter (50) and irrigated through the catheter (50). The saline drains out of the holes of distal infusion port (56) that lie along the elongated body or shaft (12), and flows around shaft (12) proximally (closer to the user of the catheter) adjacent to the balloon. This provides an isolated saline wash on the urethra side of the catheter. The fluid bathes the urethra, and drains towards the tip or glans (4) of the penis. Here, the inflow or “aspiration” holes of the distal collection port (58) lie. The injected fluid drains inwardly through these holes, back into the catheter (50) through the aspiration or collection lumen (38) after bathing the urethra (6).

The selective urethral infusion, such as just described, gains particular benefit where the wash contents are collected via collection ports also on the catheter within the urethra, such as according to the embodiments just described. However, it is to be appreciated in many applications that the irrigated wash material may flow out from the urethra through the penis tip and then be collected such as by a spill container. However, while this may be useful and still considered a benefit over conventional techniques, specimen can be lost or otherwise contaminated. Thus the provision for local collection via the catheter within the urethra is considered particularly beneficial.

Further to the present embodiments for selective urethral wash and collection, the distal (or upstream) location of the distal infusion ports relative to the more proximal (or downstream) location of the distal collection ports heretofore described for the present embodiments represents a natural downstream flow along the urethra from infusion to collection. However, it is also to be appreciated that the relative location of infusion and collection may be modified according to one of ordinary skill as further contemplated embodiments and without departing from the intended scope hereunder. This may include for example transposing their relative locations with infusion ports located proximally and collection ports located distally closer toward the bladder, or for example by alternating them in the setting of multiple ports serving each function along the length of the isolated urethra.

Moreover, in order to still further isolate the infusion and collection of selective urethral wash, additional isolation against flow around the catheter and out from the urethral region of interest may be provided proximally downstream of the distal infusion and collection ports in the urethra.

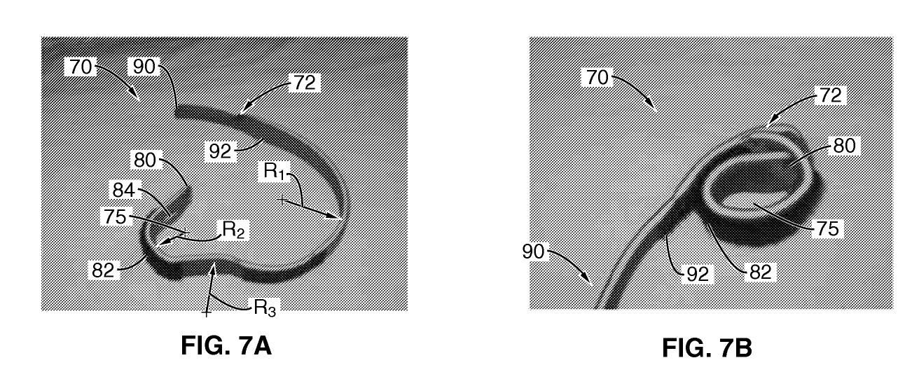

This is accomplished according to one particular embodiment by use of a penis clamp positioned around the outside of a penis when a urethral catheter is used and positioned, such as shown in FIGS. 7A and 7B. Various commercially available clamps may be employed, or otherwise manufactured to particular specifications, for this purpose and are contemplated under the intended scope of the embodiments and broad aspects hereunder. However, one such exemplary penis clamp that is considered particular beneficial for inclusion in the systems and methods herein contemplated is shown in FIGS. 7A and 7B.

More specifically, FIG. 7A shows penis clamp (70) that includes a strap (72) that extends between two opposite free ends (90) and (80). Penis clamp (70) is shown in FIG. 7A in a relaxed memory condition, with a shape adapted for manipulation for enhanced conformity to wrap around a penis and tighten down to a deformed condition with good circumferential pressure over a range of sizes. This relaxed condition has a general radius R1 of curvature shown along a significant portion of the length of the strap (72) that translates around a partially enclosed inner space (75) between ends (80,90). Another recessed area along the strap (72) is shown at radius R2 in a region adjacent to free end (80). This particular feature adjacent this end (80) provides a smaller radius for R2 than R1 which is located toward the more intermediate and longer sweeping portion of the strap (72). In general, the recess with radius R2 provides the region of inner space (75) where a penis would be housed when constricted with the strap (72), such as shown in FIG. 7B where inner space (75) is confined principally to that region.

A Velcro surface (82) is located along the strap (72) on its outside surface, e.g., the surface opposite the inner space (75), and is positioned in the embodiment shown along the shaped portion corresponding with radius R2. A second Velcro surface (92) is located along an inner surface of strap (72) at a location closer to free end (90).

In the particular embodiment shown, areas shown with radii of curvature R1 and R2 are curved in similar orientation toward the central space (75). However, according to still a further feature provided in the particular embodiment shown, a third area of inflection is also shown with a third radius of curvature R3 and that is located between the areas of curved radius R1 and curved radius R2. This area with radius R3 is inflected in the opposite direction vs. R1 and R2 that are curved around inner space (75). This puts a natural outward bias on the portion corresponding with R2 in order to form the recess that receives the penis. This provides certain benefits, such as for example without limitation enhanced securement between mating Velcro portions when wrapped in the collapsed configuration, and maintenance of round shape along region R2.

As also shown in FIG. 7A, an aperture (84) is formed through end portion (80). In use, end portion (90) is inserted through this aperture (84) to enclose the region (75). By further advancing end (90) through (84), the space (75) continues to become smaller. This allows for adjustable sizing, with substantially maintained circumferentiality, thus providing a relatively comfortable and consistently circumferential seal around a penis, such as to seal the area down around transurethral catheters as herein contemplated among various embodiments.

FIG. 7B shows an adjusted shape for penis clamp (70) that is constrained around the inner space (75) previously only partially confined by the clamp in the relaxed condition shown in FIG. 7A. Here, the inner space (75) is completely circumscribed by the strap (72), and with significantly reduced area. Moreover, the respective outer and inner Velcro surfaces (82,92) are brought into mating contact and are releasably secured together in order to remain in the shape shown until they are pulled apart.

As would be appreciated by one of ordinary skill, the strap (72) is placed around a penis of a patient in the relaxed condition shown in FIG. 7A, or perhaps even with partial outward radial expansion during application. Then, the strap is cinched together by wrapping end (90) over the outside of opposite end (80) until the enclosed inner area (75) clamps down around the penis to a desired degree of tightness. According to use with the present urethral catheter embodiments of this disclosure, this is done with such a catheter in place in the urethra and cinching the penis down over the catheter. For “downstream” isolation of the urethra around the catheter as elsewhere herein described to prevent leakage of irrigants out from the penis, a desirable location for this may be adjacent (or at the “base” of) the glans or head of the penis, such as shown at location (3) along penis (2) in FIG. 6. Here, a natural ridge at the base of the head provides further stability preventing the clamp from coming off or moving during use. Once the intended selective urethral irrigation procedure is completed, the respective Velcro portions of the strap are pulled apart and the clamp is removed.

This represents one particular embodiment herein contemplated for a “closed-loop” system and method for the isolated urethral irrigation wash and cell collection. Whereas a distal balloon on the catheter isolates the urethra from the bladder, the outer clamp isolates the region at the other end from the outside world externally of the patient. Thus by capturing the infusion outflow/irrigation ports and aspiration inflow/collection ports between these distal and proximal occlusions, the wash and collection are isolated to that region. In the still more particular illustrative embodiment shown and used in the overall system and method investigated and herein described, this circumferential clamp is made of a rubber material, e.g., a spandex-coated rubber. This assembly of inter-cooperating component parts is useful when urethral perfusion therapy and/or cell collection is being performed through a penis. In this manner, fluid does not leak around the catheter. A penile clamp that delivers a “12 o'clock+6 o'clock” squeeze (with the catheter still inside the patient) creates “dog-ears” at 3 o'clock and 9 o'clock around the catheter. While such approach may provide some benefit (versus a completely open system un-occluded at the glans), a circumferential squeeze is considered of particular benefit for a more efficient seal, and of enhanced patient comfort.

The foregoing penis clamp embodiment thus provides, among other benefits, combination benefits with the other catheter embodiments previously described above by providing a distal balloon on the catheter, and more proximally located clamp, for dual end isolation of a urethral region therebetween. This allows isolated, closed loop infusion into the urethra and collection from the area without significant loss of fluid, contamination form other areas, or contamination of the irrigant into other areas.

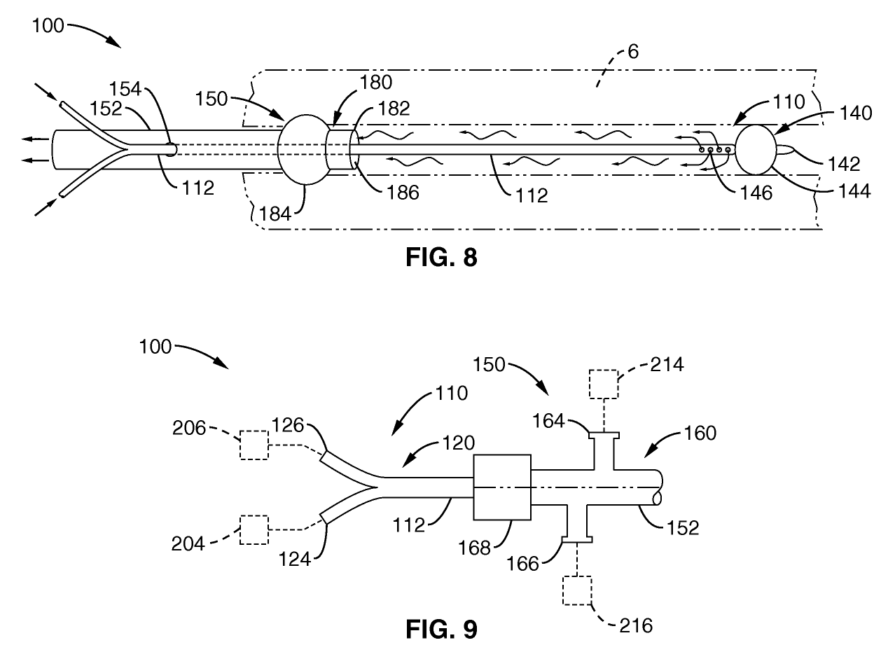

Another embodiment providing dual-end isolation is illustrated in FIG. 8. Here a catheter assembly (100) includes first and second cooperating catheters (110) and (150) as follows. Catheter (110) includes an elongated body (112) with a distal end portion (140) that includes an expandable balloon (144) proximally adjacent to a tip (142) and distal infusion ports (146) located proximally adjacent to balloon (144). Catheter (150) includes an elongated body (152) with a distal end portion (180) that includes an expandable balloon (184) located proximally adjacent its distal tip (182). However, catheter (150) includes a distal collection port (186) that constitutes an end-hole port at tip (182) of an inner lumen that is sized to slideably receive and engage elongated body (112) of catheter (110) within that lumen. In this arrangement, distal end portion (140) of catheter (110) thus extends distally from catheter (150) through distal collection port (186). In one beneficial embodiment, this is an adjustable telescoping arrangement between catheters (110) and (150) by slideable movement of this coaxial relationship. However, a fixed spacing may also work in many cases, and wherein multiple versions may be provided with different distances between balloons. Distal collection port (186) is also sized to provide sufficient clearance over and around elongate body (112) of coaxially engaged catheter (110) such that the clearance in the collection port (186) may be used to aspirate and collect the irrigated wash contents of the confined region between balloons (144, 184).

The specific embodiment shown in FIG. 8 shows balloon (184) with a larger expanded diameter than balloon (144). This is considered beneficial in circumstances for example where balloon (144) is positioned distally upstream in a ureter lumen and balloon (184) is positioned for seating at the ureteral orifice of the bladder, thus requiring a different size. However, it is also appreciated that the balloons may be of similar size, or larger distally and smaller proximally, to suit a particular purpose. The respective sizing may be accomplished by providing balloons of particular different construction, or by providing similar construction but by inflating to different parameters (e.g., different volumes).

In this present embodiment, as in others with multiple balloons, separate inflation lumens and proximal inflation ports may be provided for inflating the respective balloons. However, in multiple balloon catheters of single device construction, such as for example using one catheter body vs. two, one inflation lumen also may be used instead for expanding multiple balloons.

In addition, the expandable members provided among the embodiments throughout this description are most typically shown and described by reference to using inflatable balloons. Further embodiments contemplate that other expandable or extendable members may be used instead of inflatable balloons, such as for example expandable cages, radially deflectable or extendable walls, etc. in order to occlude areas around catheters.

The respective proximal assemblies of catheters (110) and (150) of the FIG. 8 embodiment may be provided in many different specific configurations according to one of ordinary skill, with one illustrative example shown in FIG. 8 that provides an end-to-side coupling between the devices.

However, a further example is shown for purpose of further illustration in FIG. 9. Here, proximal end portion (160) of catheter (150) is provided with a proximal inflation port (164) coupled to balloon (184) via an internal balloon inflation lumen, and with proximal collection port (166) coupled to distal collection port (186) via the thru internal lumen that houses body (112) of catheter (110). Also provided on proximal end portion (160) of catheter (150) is a third proximal port (168) that provides for proximal extension out from catheter (150) of the proximal end portion (120) of catheter (110). This may be provided as (or in conjunction with) a hemostatic valve, which may be of a type that is commercially available, such as for example a “Touhey Borst” valve, or of customized construction for a particular purpose. Such a valve may be incorporated with the catheter, or may be provided separately for adaption to a mating adaptor on the catheter port. Such hemostatic coupling is desirable, because the inner lumen corresponds also with the collection of irrigated wash contents through port (164) and thus should be closed at this interface when collecting such materials but reasonably loose or open during slideable movement between the catheters.

As also schematically shown in FIG. 9 for further illustration, various other components are contemplated for interfacing use with the embodiment shown and described, including for example: balloon inflation source (204), such as a syringe, coupled to port (124) for inflating balloon (144); infusion source (206) coupled to port (126) for infusing material into the urethra via infusion ports (146); balloon inflation source (214) coupled to port (164) for inflating balloon (184); and collection container or depository (216) coupled to port (166) for collecting selective urethral wash contents via distal collection port (186). These schematically shown cooperating assemblies may also be incorporated for cooperative use with the other embodiments herein shown and/or described.

Further to the description of the present embodiments for telescoping arrangement between inner and outer catheter members just provided, a device and method is provided by which to accomplish closed-system perfusion of a closed and/or tubular space within the body (e.g. vessel or ureter). A triple-lumen single (or double) balloon catheter may also be employed to accomplish the objective of double-sided isolation, such as according to other embodiments herein described. However, catheter embodiments with three lumens may require a relatively larger outer diameter than other approaches. While it is certainly possible to manufacture such a catheter (three lumens, and 2 balloons), diameter size optimization remains a pervasive goal such that alternative methods to accomplish closed-system perfusion of tubular body structures may provide further benefit. For applications in particular smaller or more challenging to reach areas of lumens or ducts, such as for example the ureter vs. urethra, certain design considerations may tip the scales even further toward optimizing certain particular features such as size and ability to navigate to remote locations.

Accordingly, it is a contemplated benefit to provide an ability to selectively perfuse the ureter (for example) with a chemotherapeutic drug. It is a further contemplated benefit to do so while preventing the chemo irrigation fluid from bathing the kidney, which lies just upstream (relative to physiologic urine flow) from the distal-most part of the device in the ureter. It is still further desirable to prevent the irrigation fluid from bathing the bladder, which lies just downstream (relative to physiologic urine flow) from the lower aspect of the ureter. Accordingly, an occluding balloon (on a catheter) located at the proximal and distal-most limits of the ureter can prevent fluid from flowing either proximally or distally.

According to the particular embodiment thus for perfusing the length of the ureter using a catheter in a continuous, closed system fashion, the fluid inlet and outlet open into the lumen of the target area (ureter). In using a single catheter system, three to four lumens may be employed depending upon the particular embodiment used: 1. irrigation fluid inlet, 2. irrigation fluid outlet; 3. Balloon #1, 4. Balloon #2. In one further embodiment, however, both balloons can be filled with the same lumen, and then accordingly 1 less lumen is required in this setting (2-3 lumens). Moreover, if the chosen method of perfusion is not continuous but instead “cycled” (e.g. irrigation inflow occurs, followed by irrigation outflow, both occurring through the same lumen), then 1 less lumen is also needed (2-3).

Other embodiments of the present disclosure offer multiple bases for systems and methods to be put to beneficial uses intended, including 2 to 4 lumen catheters with 1 to 2 balloons that offer either continuous or cycled close-system perfusion of a tubular structure. Presented by the telescoping arrangement of catheter components, however, is a still further unique system and method by which to accomplish select closed-system continuous perfusion of an isolated segment of a tubular body structure, by means of a “telescoping” catheter system that employs two double-lumen, single balloon catheters.

The perfusion catheter presently contemplated according to the current embodiment comprises two double lumen single balloon catheters of different outer diameters, such that one of them (which may be for example about 5 Fr outer diameter) can fit into the lumen of a larger one (which may be for example about 10 Fr outer diameter). Both possess a balloon at or adjacent to their distal tip. By providing this assembly of such construction and materials as appropriate for transureteral use, novel applications can be performed with significant positive impact on patient care associated with certain conditions in this very unique anatomical area of the body.

Further description of the transureteral use presently contemplated is provided as follows in order to present a complete understanding of this embodiment. To perfuse the ureter for example, the smaller catheter is passed through the larger one. Both are advanced as a single unit into the bladder and chosen ureteral orifice (opening of the ureter in the bladder wall), endoscopically over a guide wire (through the urethra, using a flexible scope with a light and camera). The smaller catheter is advanced far up the ureter, until its tip lies at the proximal-most limit of the ureter. Here, the smaller catheter's balloon is inflated. The larger catheter is advanced into the ureter just 1-2 cm, so that its tip rests at the distal-most limit of the ureter. Here, its balloon is inflated. By providing two catheters whose outer diameters differ by 3-4 Fr., naturally, there will be a gap between the two catheters where the smaller one enters the larger one. This gap serves as the outflow channel. Fluid cannot flow downstream past the gap opening between coaxial catheter bodies, because the balloon of the larger diameter catheter provides a complete seal circumferentially around the ureteral wall. The only place for fluid to flow is through the gap, into the larger catheter.

The perfusion irrigation fluid inflow to the region is provided by the smaller diameter catheter. It is to be appreciated that this smaller catheter may be, for example, similar to that provided elsewhere hereunder such as the embodiments of FIGS. 2A-4 (at least to the extent providing a distal balloon and distal infusion ports for isolated infusion proximal to that distal balloon). This catheter has the following design features in one embodiment considered to be of particular benefit, but not intended as limiting to the broader aspects herein contemplated: relatively small perforating holes along the 2 cm of the distal shaft of the catheter (i.e. immediately proximal to the balloon). A balloon which, when inflated radially occludes the tubular structure it lies within. The balloon described above also serves to occlude the lumen of the catheter segment distal to the balloon, so that irrigation inflow exits ONLY through the small perforating holes proximal to the balloon, and NOT through the distal end of the catheter. This may provide a closed end system distal to the balloon by fixed construction and design, or may be modified to accomplish this in an otherwise end-hole design, or may employ certain valving such as also herein contemplated elsewhere hereunder this disclosure.

The irrigation outflow is provided by the space-gap at the junction between the narrow diameter and wider-diameter telescoping catheters at the distal end of the target site (e.g. distal ureter).

As described above, the smaller catheter may “enter” the larger diameter one. Furthermore, it is contemplated that the smaller catheter in many cases preferably enters the larger catheter as far distally (outside the body) as possible. In one embodiment shown in FIG. 8, an “end to side” engagement between the catheters outside the body is employed (i.e. this is where the end of the smaller catheter enters the side aspect of the larger one, so that the distal ends of the smaller and larger catheters are totally separate.) This is useful so that the irrigation outflow (larger diameter catheter) can flow separately to a disposal bag and/or suction pump, and so that the smaller catheter can accommodate a guide wire through its central lumen.

As another embodiment, the junction point where the smaller catheter enters the larger one (e.g., in an end-to side fashion) has a ‘nipple valve” or other form of seal, to minimize the leakage of irrigation fluid from where the smaller catheter exits the larger catheter. If instead, the smaller catheter enters the larger in an end-to end fashion, then the nipple valve can fit over the lumen of the larger catheter, so that the smaller catheter fits through the nipple valve. These particular features and embodiments are illustrative, and further examples contemplated in addition to others shown and described (e.g., rotating hemostatic valve engagement for end-to-end coupling), thus demonstrating the broad scope of the overall aspect contemplated, and many beneficial further embodiments also presented.

One further example of medical use considered highly beneficial for the current “telescoping” embodiments herein disclosed is provided as follows regarding treatment of the ureter with a chemotherapeutic drug suspension. According to this embodiment, the proximal-most limit of the ureter corresponds to the distal-most limit of the catheter. After the larger diameter catheter has been advanced over a guide wire, in a retrograde fashion, through the bladder and about 2 cm for example distally past the ureteral orifice, the smaller diameter catheter is advanced through the larger one distally, until its tip resides at or closely adjacent to the distal-most limit of the ureter. Here, the guide wire is removed, and the balloon of the smaller catheter is filled with water or air, so that the ureter is occluded and such that fluid later irrigated into the distal ureter does not flow distally into the kidney. To prevent irrigation fluid from flowing distally through the smaller catheter, the catheter/balloon is designed such that when the balloon is inflated, a small segment of balloon wall occludes the main lumen of the catheter.

After the smaller diameter balloon is filled as described above, and position is confirmed radiographically or under direct vision ureteroscopically, then when the wider-diameter balloon positioned at the proximal limit of the ureter, its balloon is filled, so that irrigation fluid cannot flow distally around the catheter and into the bladder. The segment of ureter that now lies between the distal balloon (small diameter catheter) and proximal balloon (larger diameter catheter) is now effectively a closed space. Perfusion of the treatment fluid (e.g. chemotherapeutic drug suspension) through the smaller diameter catheter is initiated. The irrigation fluid can only exit the smaller diameter catheter through the small perforation holes that lie just proximal to the balloon. The irrigation fluid exits here, and passes distally along the ureter. Because the ureter is occluded both proximally and distally, the only exit site for the irrigation fluid is through the space-gap between the smaller diameter catheter telescoping distally through the larger diameter proximal catheter. To aid in the circulation of fluid from the distal ureter proximally into the large-diameter proximal catheter, the large diameter proximal catheter can be connected for example to a gentle intermittent suction pump. However, it is to be appreciated that so long as a requisite pressure drop exists in the correct direction relative to the respective fluid flow lumen, fluid will flow therethrough; such may not in certain circumstances require such applied vacuum pressure.

Other occlusion balloon catheters that have been otherwise disclosed for delivery over a guide-wire, and even to the extent intending two such catheters used together with a larger diameter one passed over a smaller one, present certain shortcomings overcome by the present disclosure, and have been adapted and intended for different purposes than those now contemplated hereunder. The particular purposes and catheter constructions and adaptations required to achieve them are particularly unique in various of the intended uses herein described. For example, the hubs of many occlusion balloon catheters are not removable, thus precluding passage of a smaller catheter into a larger one, while still being able to access the hub of the larger catheter. Furthermore, a catheter that can be introduced into another catheter in an end-to-side fashion is considered of particular benefit in certain applications, as herein described for certain of the present embodiments. Furthermore, other occlusion balloon catheters are not generally suited or intended for the lumen that accommodates the guide-wire during passage to be occluded after placement, so that irrigation may be passed through this lumen for irrigation only to the length proximal to the balloon, such as is described in further more detailed embodiments as to this beneficial feature immediately below.

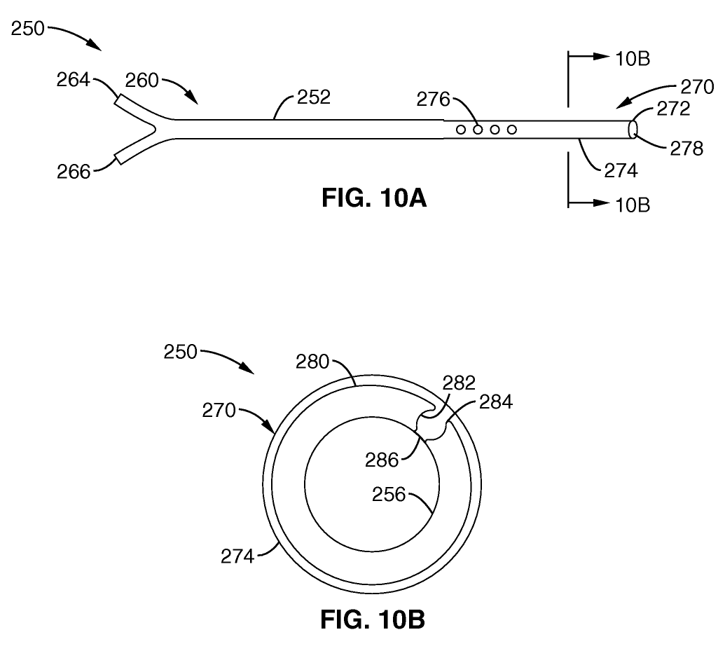

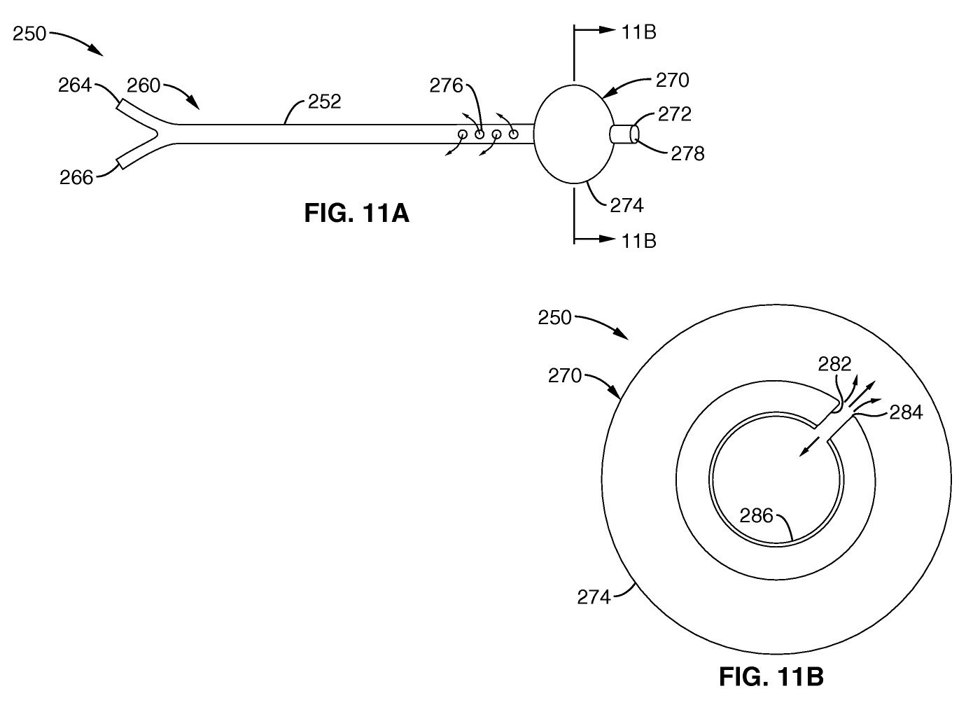

Another aspect of the present disclosure is illustrated in various modes in FIGS. 10A-11B as follows.

FIG. 10A shows a catheter (250) with an elongate body (252) extending between a proximal end portion (260) and a distal end portion (270). Distal end portion (270) includes an inflatable balloon section (274) proximally adjacent a distal tip (272), distal infusion port (278) distal to balloon (274), and intermediate infusion port (276) located proximally of balloon (274). Proximal end portion (260) includes a proximal inflation port (264) fluidly coupled to balloon (274) for inflation, and proximal infusion port (266) generally coupled to each of distal infusion port (278) and intermediate infusion ports (276) via an internal infusion lumen. Whereas FIGS. 10A-B correspond to this catheter (250) with balloon (274) deflated, FIGS. 11A-B correspond to this same catheter (250) with balloon (274) inflated.

Further to this present embodiment, an internal valve is provided between intermediate infusion ports (276) and distal infusion port (278), and that is adjustable between open and closed conditions . In the open condition, infusion through the infusion lumen (256) (see FIG. 10B) proceeds along a substantial inner diameter for the lumen to principally eject from catheter from distal port (278), and much less out side intermediate ports (276), due to the relative significantly larger size and lower resistance to flow through that open distal end port (278). However, when the valve is adjusted to the closed position, the lumen (256) is closed off at the valve and thus can not communicate with distal port (278). Accordingly, the only distal exit from lumen (256) is through intermediate infusion ports (276). In the embodiment shown, the valve is provided in the form of a deflectable member or membrane (286) that lines the inner lumen (256) and forms a deflectable wall between a balloon inflation lumen (282) and inner lumen (256). Upon pressurization of balloon inflation lumen (282) for balloon inflation (see FIG. 11B), fluid is expelled through opening (284) into the balloon (274), but compresses inward against member (286) to deflect it across the inner lumen (256) to effectively close that lumen off at the membrane. While balloon inflation pressure exceeds pressure within the infusion lumen, the infusion lumen remains closed at the membrane. Since this lumen closure occurs distal to the intermediate ports (276), an appropriately pressurized infusion to the lumen allows for effective irrigation around the catheter through these ports proximally of the balloon (274).

Among other benefits, it is to be appreciated that this present embodiment allows for selective use of the catheter for urethral irrigation and bladder irrigation or drainage. It is also to be appreciated that various other specific forms of valving may be employed to accomplish the intended results of this particular embodiment and still remain within the intended scope of the broad aspects herein contemplated. For example, the location of deflectable wall or membrane may be different than the location of the balloon inflation port within the balloon. Still further, it is also contemplated that the present embodiment may be various combined with other embodiments also herein described, either in whole or in part, and either in its specific form herein shown and described or as appropriately to be modified in order to accomplish such combination, as would be apparent to one of ordinary skill.

The disclosures of the following issued U.S. patents are herein incorporated in their entirety by reference thereto: U.S. Pat. Nos. 6,234,995; 6,582,388; 6,695,810; and U.S. Pat. No. 6,714,823.

It is to be appreciated that the present valved lumen embodiments described above provide an occluding mechanism for the central lumen of an occlusion balloon catheter tip. Specific combinations of this feature with other features herein described across the embodiments, such as in urethral or ureteral isolated irrigation assemblies and methods elsewhere described hereunder, are clearly contemplated. However, it is also to be appreciated that the benefit provided by this feature in those combinations, though of particular benefit not heretofore previously provided in the art, are not necessarily limiting and other applications are contemplated under a broad intended scope of this description. The valved lumen aspects described hereunder provide significant benefit by incorporation into or with other systems and methods, though not specifically shown or described hereunder, as would be apparent to one of ordinary skill without departing from the broad intended scope hereof. Moreover, the specific valve structures and mechanisms described by the detailed embodiments, while also considered of particular unique benefit, may be modified in certain circumstances to other structures or mechanisms so long as one or more similar objectives as stated hereunder are met.

Not withstanding the broad scope of applicability intended with respect to the novel valved lumen catheter aspects of this disclosure, one particularly beneficial embodiment for such an occluding balloon catheter balloon tip is provided in some detail in the specific detailed embodiments shown and described here. This embodiment allows for the occlusion of the catheter main lumen at the same time that the outer occluding balloon is activated (filled). In the more specific embodiment herein shown and described, the catheter's central lumen (at the tip, where the balloon lies) has for example a 2-5 mm long x 1-3 mm wide diaphragm window made of the same material (including thickness) as the balloon wall. It is to be appreciated that, because his diaphragm connects the central lumen to the lumen of the balloon port/opening of the balloon port into the balloon, when the balloon is filled with air or water (to occlude the vessel around the central catheter), the described diaphragm also responds to the pressurized fluid by bulging inward into the central lumen, thereby occluding it. When the balloon is deflated, both the balloon and the occluding diaphragm deflate and flatten. The central lumen again becomes patent.

Alternatively, it is also appreciated that, in certain particular applications, the outer balloon may not be required. Moreover, in some particular circumstances it may in fact be preferable that the catheter/stent not possess an outer balloon. Or example, in some vascular procedures it may be considered dangerous or otherwise undesirable to inflate a balloon within a vessel, such as for example due to a perceived or actual risk of over-dilating and rupturing the vessel or desire to maintain that outer lumen around the catheter otherwise patent to flow. In such cases, only the catheter lumen-occluding balloon may be provided, e.g., without the outer occluding balloon. However, in this setting emphasizing the independent value that the occluding lumen feature provides, other features of course may be included to meet the particular objectives in a given circumstance though such may not be specifically shown or described here.

One particular further developed implementation of the foregoing embodiment is described as follows, and constitutes a still further embodiment of the present disclosure. In vascular procedures, for the lysis of clot, local irrigation with Streptokinase (or other dissolving agent) is useful. In such cases, a perforated catheter is normally advanced, over a guide-wire, past the clot. After removing the guide-wire, a “plug on a wire” is advanced into the catheter, to plug the distal catheter end-hole (which now lies past the clot). Then, the dissolving agent is irrigated through the catheter. Because the distal end is plugged with the “plug on a wire”, the irrigation fluid can only escape through the perforation holes that lie proximally, along the desired length of the catheter, to bathe the clot and dissolve it. With the lumen occluding features of the present embodiments, as herein further developed in additional forms, the “plug on a wire” is obviated, as the distal tip end hole can be occluded simply by filling the dedicated balloon port with fluid, to fill the (inner) “lumen-occluding balloon”.

Another example and still further embodiment provides a perforated perfusion catheter using the aforementioned “inner occlusion only” or “dual-lumen occluding occlusion balloon catheter tip” embodiments. A catheter possessing the central lumen occluding mechanism is provided, such as described above, with small perforations proximal to the balloon so that the irrigation fluid instilled through the catheter's central lumen can escape proximal to the balloon.

It is to be appreciated that the current embodiments provided throughout the present disclosure can be beneficially incorporated into the manufacturing of occlusion balloon catheters for diagnostic imaging or collection of materials, as well as for selective drug therapy/drug delivery.

The following subspecialties are exemplary of settings where these embodiments may provide beneficial use: Interventional Radiology; Vascular surgery; Interventional Cardiology; Urology; Neurosurgery; Neuro-Interventional Radiology

The following description is provided for still further understanding of the present embodiments. If one wanted to insert a catheter into a large vessel (or any other tubular body space) for the purpose of perfusing the vessel segment between the catheter balloon end (distal) and the site of catheter entry into the vessel (proximal) with a drug solution, one is provided with the following options (in addition to the present embodiments described):

(1) Insert a dual lumen Occlusion Balloon Catheter (over a guide-wire) into the vessel, distally, and inflate the balloon so as to occlude the distal end of the vessel. Insert a second catheter to irrigate the vessel segment proximal of the inflated balloon of the first catheter;

(2) Insert a triple-lumen catheter over a guide-wire. Lumen #1 accommodates the guide-wire; lumen #2 connects to/fills the tip's balloon; lumen #3 opens through a port located proximally of the balloon, so that the infused irrigation fluid enters only the vessel segment proximal to the balloon.

(3) Same as (1) above, but a second catheter is not necessary because the catheter is fitted with one of two devices that allow the catheter lumen to occlude or be occluded after the guide-wire is removed, following standard catheter insertion over the guide-wire. One such device is a self-closing aperture end (on the catheter's distal tip), so that once the guide-wire is removed, the aperture closes, so that when the lumen is irrigated with solution, the solution cannot flow out of the catheter's distal end, but must instead exit through perforations from the lumen and through the catheter wall proximal to the balloon. One example of such a self-closing aperture is use of a grommet valve or O-ring, typically constructed of silastic, rubber, or other flexible or compressible material that has a resting memory condition where its aperture is closed off, but where the wall compresses open under presence of a shaft or device extending within or through the aperture.

(4) Another device that accomplishes the same effect as #3 is an insertable occluding-tip device for the catheter. After the guide wire is removed (following placement), an occluding-tip (connected to a semi-rigid trocar) is inserted through the proximal (extracorporal) end of the catheter to occlude the distal tip.

The foregoing approaches may provide viable options in certain circumstances or applications. However, it is also believed that each presents certain unique considerations that may manifest as shortcomings limiting their utility in certain other circumstances or applications.

For example, as option number (1) above uses two separate independent catheters, this may be particularly cumbersome, may require a separate vessel puncture for each, or a large puncture may be required if a larger catheter is to be fitted over a smaller catheter of the pair.

As another example, the option number (2) above uses a triple lumen catheter. The limitation of the triple lumen catheter approach is that, as in option number (1), an additional and separate lumen is used to accommodate both the guide-wire and the irrigation. This extra lumen occupies space, and limits the new lumen cross-sectional area of the irrigation lumen (which thus limits the volume of irrigation that can be delivered per unit time at low pressure), or requires larger size for the catheter with concomitant increase in puncture size and potential issues with increased insult to the luminal space intervened.

The limitations of the cooperating dual-device approach described in (3) are as follows. In one regard, an aperture fitting that closes after the guide-wire is removed may or will not allow the guide-wire to be easily reinserted, if at all, once it has been removed. Thus, if the position of the catheter/balloon must be adjusted after the guide-wire has been removed, the entire catheter must be replaced. Other limitations that may be associated with such a device (insertable occluding tip) include as further examples:

1. The insertion of the occluding tip can be cumbersome;

2. Correctly seating the occluding tip into the catheter tip deep within the patient, so as to ensure adequate occlusion of the catheter tip, can be difficult if the trocar or semi-rigid wire that the tip is inserted on is bent or mal-positioned;

3. To ensure that the occluding-tip is correctly positioned and is truly occluding the catheter tip prior to proceeding with irrigation through the catheter, a contrast study is often done, whereby contrast is irrigated through the catheter and fluoroscopic images are recorded. The drawback to this is obvious: increased time, potential toxicity from the contrast, and increased radiation exposure from the fluoroscopic imaging; and

4. The occluding tip can potentially “catch” on any perforating holes.

It is believed that the foregoing limitations described with respect to the other approaches described immediately above are overcome by the unique particular embodiment herein shown and described that provides for isolated irrigation through intermediately positioned irrigation ports around a catheter proximally adjacent to an inflated balloon by use of a valved catheter infusion lumen which collapses an internal membrane to close the infusion lumen off to distal infusion ports distal of the balloon upon inflating the balloon.

Various of the embodiments presented by the current disclosure offer physicians a means by which to advance a dual lumen specialized occlusion balloon catheter into position, over a guide-wire. According to certain such embodiments, upon removing the guide-wire, as the (tip's) balloon is filled, the same balloon also occludes the central lumen of the catheter (through-which the guide-wire was initially passed). Thus, as herein described the central lumen of the catheter can then be used to selectively irrigate the vessel segment proximal to the balloon. The following benefits, among others, are thus provided by these features just described: 1. Only a single catheter is necessary for distally isolated infusion; 2. As only 2 lumens are used, this design maximizes available cross-sectional area; 3. Occlusion of the catheter's central lumen is reversible; 4. The mechanism of the device is simple; 5. In case of malfunction (whereby the balloon cannot be deflated), the balloon can be popped easily simply by advancing a stiff-wire tip through the catheter.

It is to be appreciated that, while certain particularly beneficial therapeutic aspects are also elsewhere herein described and considered of independent value, the significant benefits of the diagnostic aspects of the present disclosure are also of particular significance. In addition to providing the present catheter embodiments of highly unique design for specifically performing selective urethral perfusion, the ability to provide an overall diagnostic system and method presents a highly unique and valuable result. When placed into the urethra, the urethra is irrigated through the catheter, and the fluid is collected, to serve as a diagnostic specimen. Because the saline made no contact with the bladder, it may be expected that there shouldn't be any mucus present. In actuality, however, in many cases there still is some mucus, but it is believed that this mucus and debris seen along urethral linings is from mucus/debris that came from the neobladder, and coated the urethra. However, when the quality of the voided neobladder specimen from conventional systems and methods is compared with the quality of a specimen obtained according to the embodiments of the present disclosure, it is readily apparent that the present, improved system and method offered presents a much “better” specimen. This is because the urethra has very little of the bladder's mucus.

Accordingly, the present disclosure includes various aspects for a diagnostic system that includes certain catheter-based delivery modes in combination with certain particular wash materials coupled to the catheter in a configuration adapted for cellular collection for diagnosis, and also in further combination(s) with certain diagnostic instruments, such as for example cytopathology instruments such as a microscope. Various method aspects are also herein described and are also contemplated as presenting highly beneficial, improved diagnostic techniques. In addition, it is further contemplated as a further aspect that a system may include an apparatus constructed according to the present embodiments that is beneficially packaged, typically in sterile form (though may be in a form for subsequent sterilization), together with “instructions for use”. These instructions, according to further modes and embodiments, teach how to use the packaged apparatus, which may include certain combinations with such irrigant(s) or diagnostic instrument(s), in order to perform the highly beneficial and improved diagnostic methods herein contemplated.

It is also contemplated that various of the catheter embodiments herein described are highly beneficial, novel, and of particular use unique to the industry and prior disclosures. However, in the event the particularly beneficial method aspects herein described were performed by different device implements than those specifically herein described or shown, such use is nonetheless still contemplated within the broad intended scope of the present method aspects.

The presently described systems and methods are considered to present a highly beneficial potential impact to patient care. For example, for 100 patients who have had their bladder removed, all of them (100/100) are typically indicated to require twice annual diagnostic cytology performed. Thus, a significantly improved system and method such as herein described may potentially be used with beneficial impact to each of these patients, multiple times over. Conversely, whereas therapeutic devices and their applications in providing therapy is of critical importance, of 100 people that have had their bladders removes, about 95/100 (or about 95%) will generally never have a urethral recurrence. The other about 5/100 (or about 5%) will have a recurrence.

Thus, various present aspects provide systems and methods for improved drug therapy, and safely, to these patients. However, the overall regular impact on patient care is expected to be especially widespread and to affect a great many more patients and healthcare providers via application of the diagnostic aspects of the present disclosure.

Various of the catheter embodiments herein described, though of beneficially unique design, may be of similar construction with respect to materials and manufacturing techniques as those used in conventional transurethral catheters. Accordingly, it is expected that the devices may be made at relatively low cost commensurate, or at slightly higher premium, to existing devices. Moreover, the methods for use described are considered to provide fast, reliable, and unambiguous diagnostic results. Accordingly, despite the significant steps forward provided to medical care, the methods herein described should be easily adopted into current practices without requiring significant training.

While many different patients may be diagnosed or treated with the present devices, systems, and/or methods, typical exemplary patients are those who have had their bladder removed and replaced with one made of intestine, e.g., bowel. This is a significant, sizeable population. Moreover, in this setting each patient will be generally required to have this done within 1 month. Accordingly, this represents a high potential frequency of use.

Clinical Trial #1: Transurethral Wash, Collection, & Cancer Diagnosis

Cytopathologists describe decreased diagnostic sensitivity of neobladder specimens. The purpose of this clinical trial was to compare diagnostic quality of voided (neobladder) urine specimens for cytology analysis in diagnosing bladder cancer recurrence versus selective saline urethral wash specimens acquired via certain novel embodiments of the present invention. The present trial was performed clinically on patients under IRB approval and patient informed consent. No significant safety risk was considered to be presented by the novel technique evaluated versus the comparison urine void approach, whereas improved diagnostic quality of cytology for cancer diagnosis was expected from the new technique with potentially significant clinical benefit in patient management and care.

Commercially available “Foley” type catheters were used, as modified in a similar manner and according to the embodiments as described and shown hereunder by reference to FIGS. 2A-6. The methods of use were also similar to the methods described hereunder by reference to FIG. 6. More specifically, the present experiment was conducted infusing saline through the distal infusion ports into the urethra proximally adjacent to an inflated balloon at the urethral-bladder interface, with downstream collection of the isolated urethral wash contents through the respective distal collection ports of the catheter located further downstream in the urethra toward the penis tip. In addition, an adjustable penis clamp similar to that shown and described by reference to FIGS. 7A-B was used for dual isolation of the urethra in a closed-loop infusion/collection system.

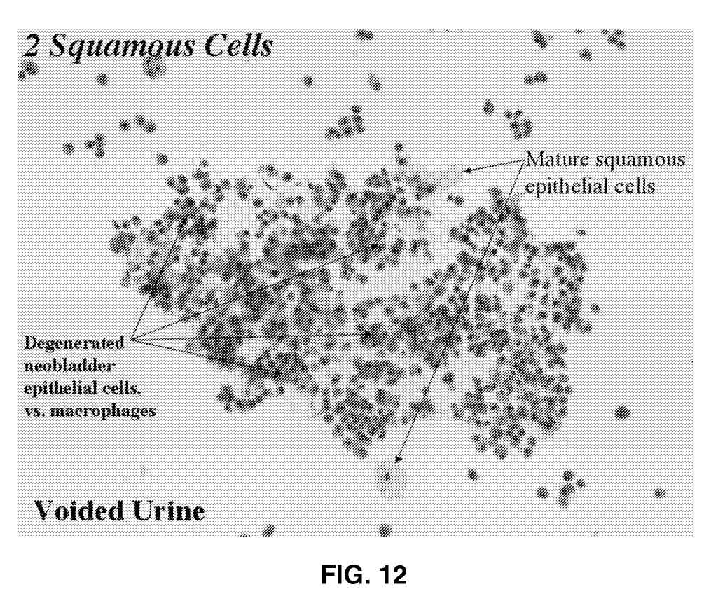

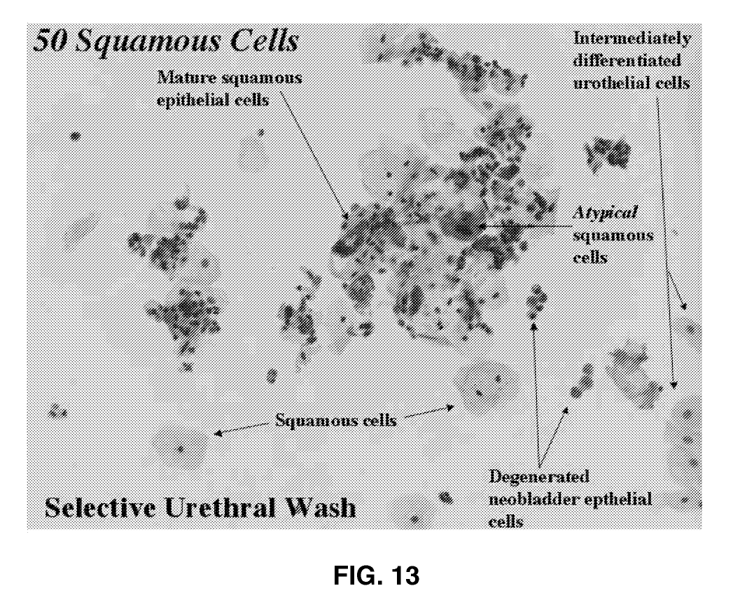

Shown in FIG. 12 is a microscopic photograph of one patient's collected specimen using the standard urine voiding technique from the neobladder. Shown in FIG. 13 is a microscopic photograph of urethral wash contents obtained with the novel modified catheter and technique, illustrating results achieved according to one particular exemplary embodiment of certain aspects of the invention. The two compared micro slides show different numbers of squamous cells. In fact, analysis of the specimens yielded the following different distributions re: the type and utility of their contents:

Voided Urine Specimen:
a. 2% Urothelial/squamous cells
b. 98% Degenerated (non-useful) cells

Selective Urethral Wash:
a. 70% Urothelial/Squamous Cells
   →30% Urothelial Cells
   →40% Squamous Cells
b. 30% Degenerated Cells

Obviously, since cancer will develop in the urothelium/squamous cells, it is necessary to fully evaluate each cell. In the sample collected with the current improved system and method, 50 squames are seen in the field. This result provides an exemplary account for the diagnostic benefit of the present embodiment. In the other comparison group (voided urine using conventional technique), much fewer squamous cells (1-2) are seen. This is also a cloudy “debris-filled” specimen. The sensitivity of cytologic analysis and cancer diagnosis is limited because the cells of interest are difficult to see.

Various different types of materials are contemplated for manufacturing the catheters and various components herein described. In general, transurethral use as contemplated will typically involve highly flexible constructions for patient comfort and conformability to the various structures involved, such as for example latex, silicone, rubber, and/or polyurethane materials. Balloons for inflation within the bladder or urethra will generally also be of the compliant, low pressure type, typically volumetrically filled and expanded at low pressures.

Clinical Trial #2: Urethral Chemotherapy Perfusion & Lavage

Devices similar to those used and described above in Clinical Trial #1 were used in this trial, except in a different overall assembly and use intended to locally deliver BCG in a closed loop fashion in the Urethra for cancer therapy.

1. Introduction and Objective:

Close follow-up with urethral lavage is recommended for patients at high risk for recurrence of bladder cancer after radical cystectomy. Where urethral cancer recurrence happens, the conventional standard of management is urethrectomy, or removal of the urethra. This results in major adverse lifestyle impact and surgical morbidity, including un-diversion of orthotopic neobladder where urine voiding is not controlled and external urine collection bag must be worn. Other management choices in setting of recurrence may include urethral sparing approaches, such as surgical resection and surveillance. However, this is not feasible where there is no distinct target for resection, such as in the case of biopsy being positive for carcinoma in-situ, or negative biopsy.

Limited reports suggest that select pathologic types of urethral recurrence can be managed with urethral therapy. Certain particular previous work has suggested that intraurethral perfusion therapy of Bacille Calmette Guerin (BCG) is a viable treatment alternative to primary urethrectomy for carcinoma in situ (CIS) urethral recurrence following radical cystectomy. Of 10 patients observed in one study, 6 were positive for carcinoma in situ with denuding urethritis, with 4 presenting with papillary carcinoma. Five of six of the CIS patients, or 83%, were considered successful with mean survival of 84 months and a range of (14-122 months). Of the Papillary group, zero of four, or 0%, were considered successful with mean survival of 44 months and a range of (12-91 months).

The disclosure of the following publication is herein incorporated in its entirety by reference thereto: Varol, C. et al., “Treatment of urethral recurrence following radical cystectomy and ileal bladder substitution.” J Urol. 2004 September; 172(3):937-42.

However, selective perfusion of the urethra, either to deliver BCG chemotherapy, or to obtain urethral cytology, is technically challenging. Spillage of BCG onto the patient's skin and elsewhere poses a significant exposure hazard to the patient and administering health professional. A closed-system low-pressure method to perfuse the urethra is needed to minimize chemotherapy exposure risks and to facilitate diagnostic urethral lavage.

Accordingly, the purpose of the present clinical trial was to evaluate use of such a system, provided according to certain aspects of the present embodiments, to confirm safety and efficacy in treating urethral cancer recurrence

2. Methods:

Two patients with suspected urethral transitional cell carcinoma (TCC) following previous radical cystectomy and orthotopic neobladder replacement were given pre-treatment intraurethral diagnostic regimen as described in Clinical Trial #1, in addition to cystologic biopsy. One of the two patients was diagnosed with denuding urethritis, with biopsy not revealing cancer. The other patient presented with superficial papillary tumor, with positive biopsy. They were then treated with intraurethral BCG perfusion therapy weekly for 6 weeks, followed by diagnostic urethral lavage 6 weeks later.

A third patient, who had previously undergone radical cystectomy with ileal conduit urinary diversion was also suspected of having urethral TCC, and underwent diagnostic urethral lavage only.

A standard latex 16 Fr. 3-way Foley catheter was modified to deliver irrigation selectively to the urethra, as shown in FIGS. 4A-5 and illustrated in FIGS. 2A-3B. No fluid was allowed to drain from the isolated region of urethra into the bladder due to expansion of the distal balloon of the urethral infusion and collection catheter, nor to leak past the urethral meatus due to use of a penis clamp similar to that shown and described by reference to FIGS. 7A-B. Irrigation fluid was delivered through the catheter, allowed to bathe the urethra, and then collected from the catheter in a closed-system such as described and shown in FIG. 6.

For each chemotherapy treatment, a standard 100 ml mixture of BCG and saline was infused. Diagnostic urethral lavage was performed by infusion of normal saline only, and according to the same catheter using methods previously described above such as for Clinical Trial #1. Irrigation was delivered by gravity into the catheter at 20 cm H20 pressure.

One day after treatment for each patient, post-treatment diagnostic selective saline urethral wash was performed, also according to similar devices and methods as the pre-treatment diagnostic regimen.

3. Results:

In all treatment sessions (15/15) the final volume of irrigation delivered exactly equaled the collected outflow. In all sessions, there was no leakage of irrigation around the urethral meatus, which remained dry throughout. The procedure was well tolerated by all three patients and there were no complications.

Mean follow-up was 10 months. Post-treatment cytology was performed at 3 and 6 months and ws negative. Post-treatment urethroscopy at 4 and 6 months also were negative. At 10 month follow-up, one patient was alive and demonstrating a durable response, whereas the other patient was deceased with brain metastastis at 7 months.

4. Conclusions:

The proposed closed-system perfusion catheter is simple to construct using available 3-way Foley catheter products, and offers a safe, simple, and reliable method by which to administer intraurethral chemotherapeutic agents. In addition, urethral lavage specimens can be collected using only a minimal volume of saline irrigation and without loss due to spillage.

As illustrated elsewhere hereunder, certain present embodiments provide a closed-ended triple-lumen urethral catheter. One lumen serves to inflate a balloon at the closed-end of the catheter, and the other two lumens serve as irrigation fluid inlet (e.g., to distal urethra), while the third lumen serves as the irrigation fluid outlet (e.g., at proximal end of urethra, but just distal to the end of the penis or glans penis).

Other embodiments provide a closed ended catheter with two balloons (one proximal and the other distal), with two dedicated irrigation lumens. The balloons can both be inflated with one inflation port (3-lumen total: double-balloon, double irrigation lumen catheter), or separate ports (4 lumen total: double-balloon, double irrigation lumen catheter). This latter device is particularly beneficially useful for (but not limited to) two highly beneficial applications:

1. Selective closed-system urethral administration of toxic chemotherapeutic drugs. The closed-system design prevents/minimizes the risk of the drug exiting the penile urethra and posing health-risks to either the patient or the administering health care worker(s) via inhalation, ingestion, or by contact exposure).

2. Obtaining selective urethral washings for cytologic analysis. The catheter device can be irrigated with plain saline, and the effluent (urethral washing) can be easily collected without risk of spillage or contamination.

Other embodiments described above can be used for a variety of other purposes in the medical arena: vascular imaging, and various surgical subspecialties, including vascular surgery, biliary surgery, general and gynecologic surgery, as may be appropriate modified and adapted from the specially adapted urethral devices and methods described.

The present embodiments provide a highly beneficial, novel device and method for use in men in particular, though not by gender limitation to the intended scope. Women are diagnosed with selective urethral carcinoma recurrence much more rarely than men. One previously disclosed device occludes the urethra proximally and distally, and is a double balloon catheter made by BARD, USA, whereby two spaced balloons are inflated to occlude the urethra at the bladder neck and at a second location along the urethra, e.g., more proximally along the catheter. This catheter, however, has a fixed spacing between balloons. Also, it provides only one lumen dedicated to irrigation (the other one or two lumens are dedicated to the inflation of the balloon). This catheter is generally intended for used to selectively inject radiographic contrast to the urethra between both balloons, such as for example to image fistulas. There is no outlet for the irrigant, such as a second dedicated collection lumen would provide according to certain of the present embodiments of this disclosure. The various combinations of features as herein provided are considered highly beneficial improvements over such other different approaches. Such combinations as providing these catheter assemblies with saline irrigation wash material, BCG, or external diagnostic equipment such as microscopes for diagnosis of collected cells for cancer, have not been previously described in such closed loop system, in particular within the urological tracts, including in particular the highly unique respective anatomies of the ureter and the urethra.

The invention has been discussed in terms of certain preferred embodiments. One of skill in the art will recognize that various modifications may be made without departing from the scope of the invention. Although discussed primarily in terms of transurethral cancer applications, it should be understood that the embodiments could be used for other applications, such as other transurethral applications, or in relation to cancer diagnosis or therapy in other body lumens or spaces. In addition, while particular cooperating or adjunctive treatment or other accessory devices are described for use in conjunction with the present embodiments, other modifications are contemplated as would be apparent to one of ordinary skill. Moreover, while certain features may be shown or discussed in relation to a particular embodiment, such individual features may be used on the various other embodiments of the invention.

The disclosures of the following issued U.S. patents are herein incorporated in their entirety by reference thereto: U.S. Pat. Nos. 4,423,725; 4,610,662; 4,625,726; 4,636,195; 4,911,163; 4,930,496; 5,002,532; 5,256,141; 5,344,435; 5,366,490; 5,462,529; 5,468,239; 5,484,412; 5,496,271; 5,588,961; 5,591,129; 5,599,307; 6,146,396; 6,165,168; 6,235,025; 6,283,940; 6,368,338; 6,447,505; 6,477,426; 6,517,534; 6,638,245; 6,702,782; 6,758,853; and 6,994,717.

The disclosures of the following U.S. patent application Publications are also herein incorporated in their entirety by reference thereto: US 2003/0195478; and US 2004/0079429.

The disclosures of the following PCT International Patent Application Publications are also herein incorporated in their entirety by reference thereto: WO 99/10038; and WO 2004/045702.

The disclosure of the following article publication is also herein incorporated in its entirety by reference thereto:

Various modifications to the current embodiments herein shown and described according to one of ordinary skill without departing from certain broad aspects herein contemplated. In one example, certain three lumen catheters are shown and described such as for one-sided isolation of infusion of irrigants into a urethra and collection. Certain embodiments shown and described are modified devices manufactured for other purposes, such as by suturing off communication to end-tip ports of infusion and collection lumens, and by forming new ports into those lumens proximally of a balloon. This, of course, may be manufactured with the desired features vs. modifying another catheter. Also, a two-lumen modification of that, and of the respective methods of use, may be made for distally isolated infusion into the urethra, and collection of effluent that exits the urethra and is collected separately from the catheter, such as by gravity collection.

This disclosure variously describes the embodiments in terms of systems, assemblies, or devices for diagnosis and treatment of cancer involving the urethra. While combinations of the components of such embodiments are highly beneficial, it is contemplated that each individual component alone may be highly beneficial, such as for example by virtue of their ability to be made and/or sold separately to be later interfaced with the other components.

The invention has been discussed in terms of certain preferred embodiments. One of skill in the art will recognize that various modifications may be made without departing from the scope of the invention. Although discussed primarily in terms of diagnosing and treating cancer, it should be understood that the embodiments could be used for other applications, such as for example related to other medical conditions associated with the same anatomical structures, or in other luminal structures in the body. In addition, while particular cooperating or adjunctive treatment or other accessory devices are described for use in conjunction with the present embodiments, other modifications are contemplated as would be apparent to one of ordinary skill. Moreover, while certain features may be shown or discussed in relation to a particular embodiment, such individual features may be used on the various other embodiments of the invention.

1. A medical device system, comprising:
a transurethral catheter assembly with a proximal end portion and a distal end portion configured to be positioned at least in part along a urethra in a patient with the proximal end portion extending externally of the patient's body;
an infusion lumen extending between a proximal infusion port along the proximal end portion and a distal infusion port positioned along the distal end portion;
wherein the infusion lumen is adapted to infuse a wash medium into the urethra via the infusion lumen through the distal infusion port; and
a collection system configured to collect urethral wash contents created by infusing the wash medium into the urethra through the distal infusion port.

2. A medical device system, comprising:
a transurethral catheter assembly with a proximal end portion and a distal end portion that is configured to be positioned at least in part within a urethra of a patient;
a distal expandable member located along the distal end portion;
a proximal expandable member located along the distal end portion proximally of the distal expandable member;
wherein the proximal and distal expandable members are longitudinally separated from each other along the distal end portion;
an infusion lumen fluidly coupled to a proximal infusion port located along the proximal end portion and a distal infusion port located along the distal end portion between the two spaced expandable members; and
a collection system that comprises a collection lumen fluidly coupled to a proximal collection port along the proximal end portion and a distal collection port located along the distal end portion between the two spaced expandable members.

3. A medical device system, comprising:
a transurethral catheter assembly with a proximal end portion and a distal end portion that is configured to be positioned at least in part within a urethra of a patient;
a luminal isolation assembly located along the distal end portion and that is configured to substantially isolate at least a region of the urethra for local fluid delivery;
a volume of wash medium coupled to the catheter in a manner configured to selectively infuse the volume into the region of urethra isolated by the luminal isolation assembly; and
a collection system configured to collect urethral wash contents created by infusing the urethral wash medium into the region of urethra isolated by the luminal isolation assembly.

4-5. (canceled)

6. The system of claim 1, wherein the transurethral catheter assembly further comprises:
a radially expandable distal member located on the distal end portion distally of the distal infusion port;
wherein the distal infusion port is located proximally of the distal member;
wherein the expandable distal member when expanded is configured to isolate the wash medium infused into the urethra through the distal infusion port to a region of the urethra located proximally of the distal member; and
wherein the collection system is configured to collect the isolated infusion of the urethral wash medium.

7. The system of claim 3, wherein the luminal isolation assembly comprises:
a radially expandable distal member located on the distal end portion; and
wherein the expandable distal member when expanded is configured to isolate the wash medium infused into the urethra to a region of the urethra located proximally of the distal member; and
wherein the collection system is configured to collect the isolated infusion of the wash medium from the region.

8. The system of claim 7, further comprising:
an infusion lumen extending between a proximal infusion port along the proximal end portion and a distal infusion port positioned along the distal end portion proximally of the distal member;
wherein the infusion lumen is adapted to infuse the volume of wash medium into the isolated region of urethra through the distal infusion port;

9-10. (canceled)

11. The system of claim 2, wherein the radially expandable distal member comprises an inflatable distal balloon.

12. The system of claim 11, further comprising:
a distal port located distally of the distal infusion port and also distally of the distal balloon;
wherein the infusion lumen is coupled to the distal port;
an adjustable valve coupled to the infusion lumen and that is adjustable between an open condition and a closed condition;
wherein in the open condition the proximal infusion port communicates via the infusion lumen with the distal port; and
wherein in the closed condition the proximal infusion port communicates via the infusion lumen with the distal infusion port but not with the distal port.

13. The system of claim 12, wherein:
the valve comprises a deflectable member located along a portion of an internal wall of the catheter assembly that separates the infusion lumen and an inflation lumen of the distal balloon.

14. The system of claim 13, wherein:
the deflectable member is positioned distally from the distal inflation port;
the valve remains open relative to the infusion lumen when the distal balloon is deflated, thereby allowing fluid communication between the distal port and the proximal infusion port;
the valve is actuatable to a closed condition relative to the infusion lumen upon pressurization of the balloon inflation lumen during inflation of the distal balloon, thereby deflecting the deflectable member into the infusion lumen to collapse across and substantially close the infusion lumen with respect to fluid communication between the distal port and the distal infusion port; and
whereby the valve in the closed configuration allows for pressurized fluid within the infusion lumen to be expelled from the infusion lumen substantially through the distal infusion port proximally of the inflated distal balloon.

15. The system of claim 1, wherein:
the collection system comprises a collection lumen that extends between a proximal collection port along the proximal end portion and a distal collection port located along the distal end portion proximally of the distal member; and
whereby the urethral wash contents created by the urethral wash medium infused through the distal infusion port are collected through the collection lumen via the distal collection port.

16. The system of claim 7, wherein:
the collection system comprises a collection lumen that extends between a proximal collection port along the proximal end portion and a distal collection port located along the distal end portion proximally of the distal member; and
whereby the urethral wash contents created by the urethral wash medium infused through the distal infusion port are collected through the collection lumen via the distal collection port.

17. The system of claim 1, further comprising:
a radially expandable distal member located along the distal end portion;
a radially expandable proximal member located along the distal end portion proximally of the radially expandable distal member;
a longitudinal spacing between the expandable distal member and the expandable proximal member configured to isolate a region of lumen surrounding the catheter between the expandable members in an expanded configuration;
wherein the distal infusion port is located between the proximal and distal members, such that an isolated region between the proximal and distal members may be selectively washed with the infused wash medium; and
wherein the collection system is configured to collect the washed contents and infused wash medium from the isolated region between the proximal and distal members in a substantially closed loop system.

18. The system of claim 17, wherein the proximal and distal members comprise inflatable proximal and distal balloons, respectively.

19. The system of claim 18, wherein the catheter further comprises:
an outer catheter member with a proximal end portion and a distal end portion and an inner lumen;
an internal catheter member with a proximal end portion and a distal end portion and that is located at least in part within the inner lumen of the outer catheter member and with the distal end portion of the internal catheter member extending distally from the inner lumen;
wherein the proximal balloon is located along the distal end portion of the outer catheter member;
wherein the distal balloon is located long the distal end portion of the inner catheter member;
wherein the inner catheter member is slideable longitudinally relative to the outer catheter member in a telescoping arrangement such that the spacing between the proximal balloon and distal balloon is adjustable.

20. The system of claim 19, wherein:
a coaxial space is located between the outer catheter member and inner catheter member, and
at least one of the infusion lumen or collection system is fluidly coupled to the coaxial space.

21. The system of claim 2, further comprising:
a radially expandable distal member located along the distal end portion;
a radially expandable proximal member located along the distal end portion proximally of the radially expandable distal member;
a longitudinal spacing between the expandable distal member and the expandable proximal member configured to isolate a region of lumen surrounding the catheter between the expandable members in an expanded configuration; and
wherein the distal infusion port and distal collection port are both located between the proximal and distal members, such that an isolated region between the proximal and distal members may be flushed with the infused wash medium and collected through the distal collection port in a substantially closed loop system.

22. The system of claim 21, wherein the proximal and distal members comprise inflatable balloons, respectively.

23. The system of claim 22, wherein the catheter further comprises:
an outer catheter member with a proximal end portion and a distal end portion and an inner lumen;
an internal catheter member with a proximal end portion and a distal end portion and that is located at least in part within the inner lumen of the outer catheter member and with the distal end portion of the internal catheter member extending distally from the inner lumen;
wherein the proximal balloon is located along the distal end portion of the outer catheter member;
wherein the distal balloon is located long the distal end portion of the inner catheter member;
wherein the inner catheter member is slideable longitudinally relative to the outer catheter member in a telescoping arrangement such that the spacing between the proximal balloon and distal balloon is adjustable.

24. The system of claim 23, wherein:
a coaxial space is located between the outer catheter member and inner catheter member, and
at least one of the infusion lumen or collection system is fluidly coupled to the coaxial space.

25-114. (canceled)
